(19) United States
(12) Patent Application Publication
(10) Pub. No.: US 2013/0317312 A1
(43) Pub. Date: Nov. 28, 2013

(54) ACTUATABLE RETRACTOR

(71) Applicant: Invuity, Inc., San Francisco, CA (US)

(72) Inventors: Robert K. Eastlack, San Diego, CA (US); Alex Vayser, Mission Viejo, CA (US)

(73) Assignee: Invuity, Inc., San Francisco, CA (US)

(21) Appl. No.: 13/900,032

(22) Filed: May 22, 2013

Related U.S. Application Data
(60) Provisional application No. 61/651,780, filed on May 25, 2012.
(60) Provisional application No. 61/660,552, filed on Jun. 15, 2012.

(51) Int. Cl. A61B 17/02

(52) U.S. Cl. CPC A61B 17/02
USPC 600/215; 600/217

(57) ABSTRACT

A positionable surgical retractor comprises a retractor blade and a coupling element. The retractor has a first surface adapted to engage and retract tissue away from a surgical field. The coupling element is coupled to the retractor blade or disposed in a wall of the retractor, and may be coupled to an anchoring element. The retractor is positionable relative to the anchor so as to engage and retract the tissue.

### CROSS-REFERENCE

The present application is a non-provisional of, and claims the benefit of U.S. Provisional Patent Application No. 61/651,780 (Attorney Docket No. 40556-725.101) filed on May 25, 2012; the present application is also a non-provisional of, and claims the benefit of U.S. Provisional Patent Application No. 61/660,552 (Attorney Docket No. 40556-725.102) filed on Jun. 15, 2012; the entire contents of each of the above referenced provisional patent applications is incorporated herein by reference.

### BACKGROUND OF THE INVENTION

Access to a surgical field often requires retraction of tissue from the surgical field to allow a surgeon to visualize the surgical field and to provide space so that the surgeon can work in the surgical field. Due to the varying anatomy of the body, many different retractors and retraction techniques have been developed. However, with the trend toward minimally invasive surgery, improved access involving smaller incisions and lower profile surgical instruments are often required. Therefore, there still is a need for improved retractors and methods of use. At least some of these objectives will be met by the embodiments disclosed herein.

### SUMMARY OF THE INVENTION

The present invention generally relates to medical devices and methods, and more particularly relates to surgical instruments such as retractors and methods of retracting tissue.

In a first embodiment, a positionable surgical retractor comprises a retractor blade having a first surface, a second surface and a wall extending therebetween. The first surface is adapted to engage and retract tissue away from a surgical field, and wherein the second surface is opposite the first surface. The retractor also comprises a coupling element coupled to the retractor blade or disposed in the wall. The coupling element is adapted to be coupled to an anchor element, and wherein the retractor is positionable relative to the anchor so as to engage and retractor the tissue.

The retractor blade may comprise an elongate tubular body and it may be a cylinder. The retractor blade may also be a waveguide for transmitting light by total internal reflection. The waveguide may comprise a light input portion, light extraction features adjacent a distal end of the retractor blade, and a light transmitting portion disposed therebetween. Light is input into the retractor blade from the light input and light is transmitted through the light transmitting portion by total internal reflection. Light is extracted and directed from the light extraction features of the retractor blade to the surgical field. The light extraction features may comprise a plurality of facets, prisms, or lenses. The retractor blade may also have an illumination element for providing light to the surgical field. The illumination element may be a fiber or a solid optical material, or an optical waveguide. Various optical coatings may be applied to any of the illumination elements in order to obtain a desired quality of light. The illumination element may be intergrated into the walls of the retractor or it may be a detachable element.

The retractor blade may further comprise a flanged region adjacent a distal end of the retractor and that is adapted to prevent tissue from sliding off the retractor. The retractor blade may also comprise a contoured distal end that is adapted to conform to anatomy in the surgical field. The contoured distal end allows movement of the retractor over the anatomy. Some embodiments may have deployable anchors such as teeth to help engage tissue. Such a feature may be pushed out from a protective sheath to expose the teeth. The teeth may or may not be fixed.

The coupling element may comprise a snap fitting that is adapted to engage the anchor or it may comprise a tubular channel. The tubular channel may be disposed on the first or the second surface of the retractor blade, or it may be disposed in the wall of the retractor blade.

The retractor may comprise a handle that is coupled with the retractor blade. The handle may be adapted to facilitate actuation of the retractor blade. The retractor blade may have an adjustable length or an adjustable width.

The retractor blade may comprise a plurality of retractor blades that are hingedly coupled together, and that have a collapsed configuration for insertion into an incision, and an expanded configuration for retracting tissue in the surgical field. The plurality of retractor blades may be adjacent one another in the collapsed configuration, and the plurality of retractor blades may be actuated away from one another in the expanded configuration. The plurality of retractor blades may form a semi-circle in the expanded configuration. In some embodiments, the plurality of retractor blades may comprise three retractor blades hingedly coupled together, and the plurality of retractor blades may form a triangle, diamond, or other polygon in the expanded configuration. In still other embodiments, the plurality of retractor blades may comprise two retractor blades each having a slot for slidably receiving a third retractor blade. The third retractor blade may hold the two slotted retractor blades in the expanded configuration. Other embodiments may have more than three retractor blades. Any number of hinges may be used between the blades, therefore any number of polygon shapes may be formed by the retractor blades when expanded about their hinges, such as a diamond shape. The larger the number of retractor blades, the closer the expanded retractor blades will be to a circle shape if desired.

In another aspect of the present invention, a system for retracting tissue in a surgical field comprises the surgical retractor described above and an anchoring element. The anchoring element may comprise a guidewire, a pedicle screw tower, or other spinal instrumentation.

In still another aspect of the present invention, a method for retracting tissue in a surgical field comprises anchoring an anchoring element in the surgical field, coupling a retractor blade to the anchoring element, and disposing the retractor blade in the surgical field. The method also comprises actuating the retractor blade about the anchoring element, and retracting the tissue in the surgical field.

The anchoring element may comprise a guidewire and anchoring the anchoring element may comprise anchoring the guidewire in the surgical field. The anchoring element may comprise spinal instrumentation, and anchoring the anchoring element may comprise anchoring the spinal instrumentation in the surgical field. The anchoring element may comprise a pedicle screw tower and anchoring the anchoring element may comprise anchoring the pedicle screw tower in the surgical field.

Coupling the retractor blade may comprise slidably engaging the retractor blade with the anchoring element or snap fitting the retractor blade with the anchoring element. The retractor blade may be releasably engaged with the anchoring element. In some embodiments, a portion of the anchoring element may be a part of the retractor. Thus, the portion of the anchor that is a part of the retractor will be coupled with another portion of the anchor which may be anchored to the tissue such as bone. The pedicle screw tower may have integrated retractor blades.

Disposing the retractor blade in the surgical field may comprise sliding the retractor blade over the anchoring element into the surgical field.

Actuating the retractor blade may comprise rotating the retractor blade about the anchoring element. Rotating the retractor blade may comprise eccentrically rotating the retractor blade around the anchoring element.

Retracting tissue in the surgical field may comprise retracting a muscle such as a multifidi or paraspinal muscle. Retracting tissue may expose a facet joint.

The retractor blade may comprise a waveguide and the method may further comprise illuminating the surgical field with light from the waveguide. In other embodiments, other illumination elements may be engaged with the retractor to illuminate the surgical field. Exemplary illumination elements include fiber optics, LED lights, or other illuminators.

The retractor blade may comprise a plurality of retractor blades that are disposed adjacent one another in a collapsed configuration, and the method may further comprise actuating the plurality of retractor blades from the collapsed configuration into an expanded configuration where the plurality of retractor blades are actuated away from one another. The method may also comprise locking the plurality of retractor blades in the expanded configuration.

These and other aspects and advantages of the present invention are evident in the description which follows and in the accompanying drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates anterior lumbar interbody fusion (ALIF).

FIG. 2A illustrates posterior fusion.

FIG. 2B illustrates a midline incision and exposure.

FIGS. 3A-3D illustrate lateral lumbar interbody fusion or LLIF.

FIG. 4 illustrates transforaminal lumbar interbody fusions (TLIF).

FIGS. 5A-5D illustrate percutaneous pedicle screw and rod placement.

FIGS. 6A-6B show posterior spinal fixation placement.

FIGS. 7A-7C illustrate various embodiments of a retractor.

FIGS. 8A-8H illustrate other embodiments of a retractor.

FIGS. 9A-9C illustrate other features on a retractor.

FIG. 10 illustrates a telescoping retractor.

FIGS. 11A-11B illustrate a retractor with adjustable channel for adjusting sweep.

FIGS. 11C-11E illustrate a mechanism for controlling rotation.

FIGS. 12A-12B illustrate retractors with handles.

FIG. 13 illustrates use of an arm to hold the retractor.

FIG. 14A illustrates engagement of a retractor to a frame.

FIGS. 14B-14C illustrate exemplary blades used with the frame in FIG. 14A.

FIGS. 15A-15C illustrate use of a retractor.

FIGS. 16A-16E illustrate use of a retractor in spinal surgery.

FIG. 17 illustrates an illuminated retractor.

FIG. 18 is a cross-section taking along the line D-D in FIG. 17.

FIG. 19 is a perspective view of a COP optical waveguide with a curved input light coupling.

FIG. 20 a perspective view of the distal end of the optical waveguide in FIG. 19.

FIG. 21 is a perspective view of an optical waveguide with a split input coupling.

FIG. 22 is a cutaway view of the embodiment in FIG. 21.

FIG. 23 is a cross-section taken along the line B-B in FIG. 21.

FIG. 24 is another embodiment of an optical waveguide with split input coupling.

FIG. 25 is yet another embodiment of an optical waveguide with split input coupling.

FIG. 26 is a cross-section of a distal portion of an optical waveguide

FIG. 27 is a cross-section of another embodiment of an optical waveguide distal portion.

FIG. 28 is a perspective view of another embodiment of an optical waveguide with reinforced and shielded split input coupling.

FIG. 29 is a cutaway view of the optical waveguide in FIG. 28.

FIG. 30 is a perspective view of the optical waveguide in FIG. 28 with the shield removed for clarity.

FIG. 31 is a sideview of the optical waveguide in FIG. 30.

FIG. 32 is a cutaway perspective view of an optical waveguide with the shield removed for clarity.

FIG. 33 is a close up front view of the input connector in FIG. 32.

FIG. 34 is a perspective view of a splittable optical waveguide.

FIG. 35 is a cutaway view of the optical waveguide in FIG. 34.

FIG. 36 is a cutaway view of an optical waveguide with an extended reflecting surface.

FIGS. 37A-37D illustrate various positions of the retractor relative to the anchoring element and surgical field.

FIG. 38 illustrates an alternative embodiment of a retractor with an outer tube disposed thereover.

FIGS. 39A-43D illustrate exemplary embodiments of expandable retractors.

FIGS. 44A-44F illustrate exemplary blade configurations.

### DETAILED DESCRIPTION OF THE INVENTION

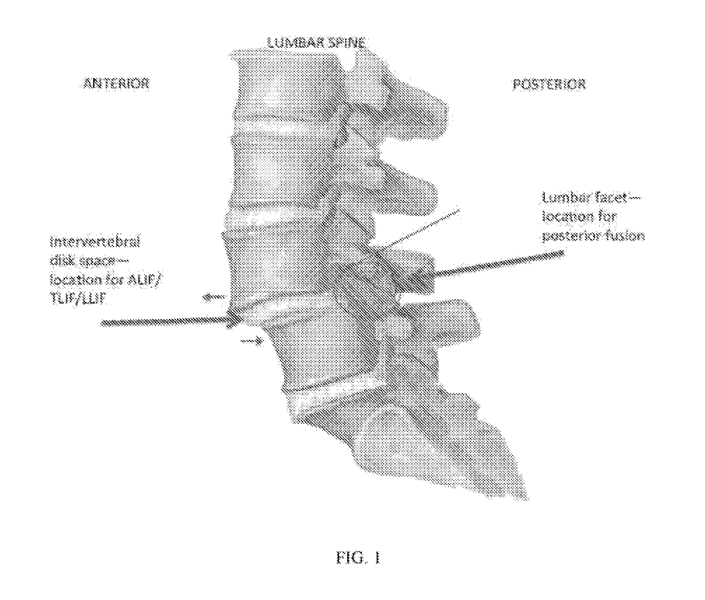

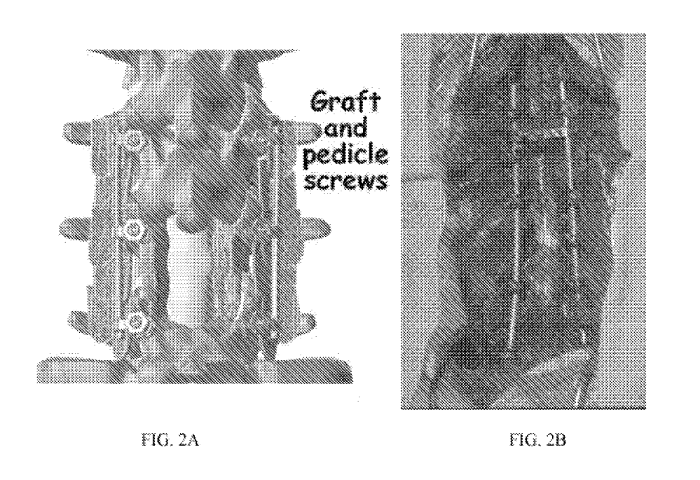

Various exemplary embodiments of retractors and their use are disclosed herein. Any of the features may be substituted or combined with features from other embodiments disclosed herein. Additionally, use of the retractors will be described with emphasis placed on retraction of tissue during spinal surgery, however this is not intended to be limiting and the retractors may be used wherever tissue retraction is required. Spinal fusion is a surgical approach for treating pain and deformities in patients with a number of back related diseases including scoliosis, disc herniation, degenerative disc disease, kyphosis, spondylolisthesis, etc. During fusion, two or more vertebrae are joined together. Facet fusions have long been a component of successful posterior spinal arthrodesis, particularly with adolescent scoliosis corrections. FIG. 1 illustrates posterior spinal fusion utilized with ‘360-degree’ fusions, in which anterior lumbar interbody fusion (ALIF) and posterior lumbar fusions illustrated in FIG. 2A are performed through traditional open approaches, as well as with posterior cervical fusions.

All of these facet or posterior fusion procedures have traditionally been performed through standard open surgical approaches, whether this has been in the cervical, thoracic, or lumbar spine regions. Classically, such open techniques are completed through a midline incision and exposure as seen in FIG. 2B, and ultimately via direct visualization.

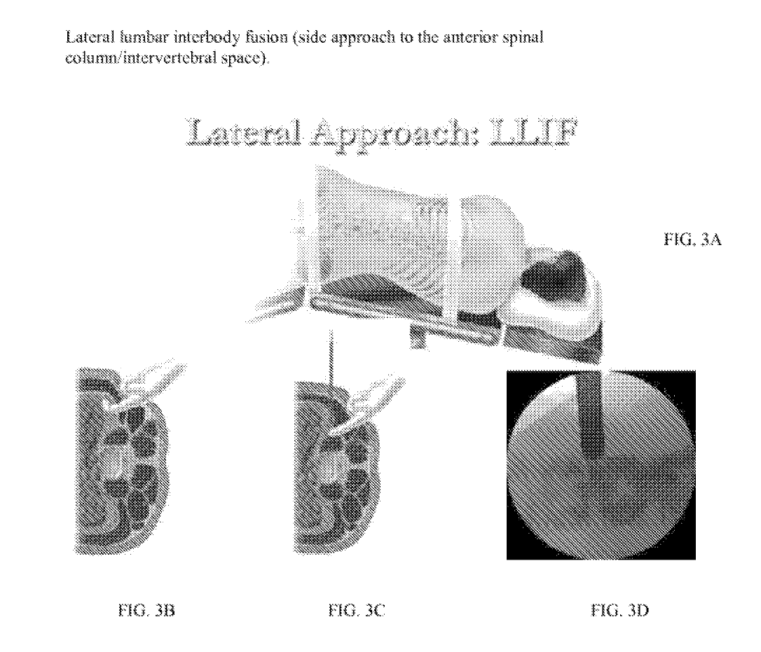

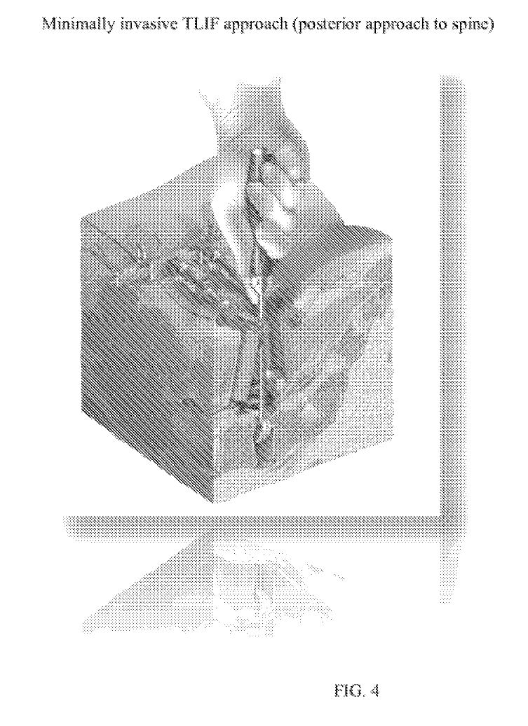

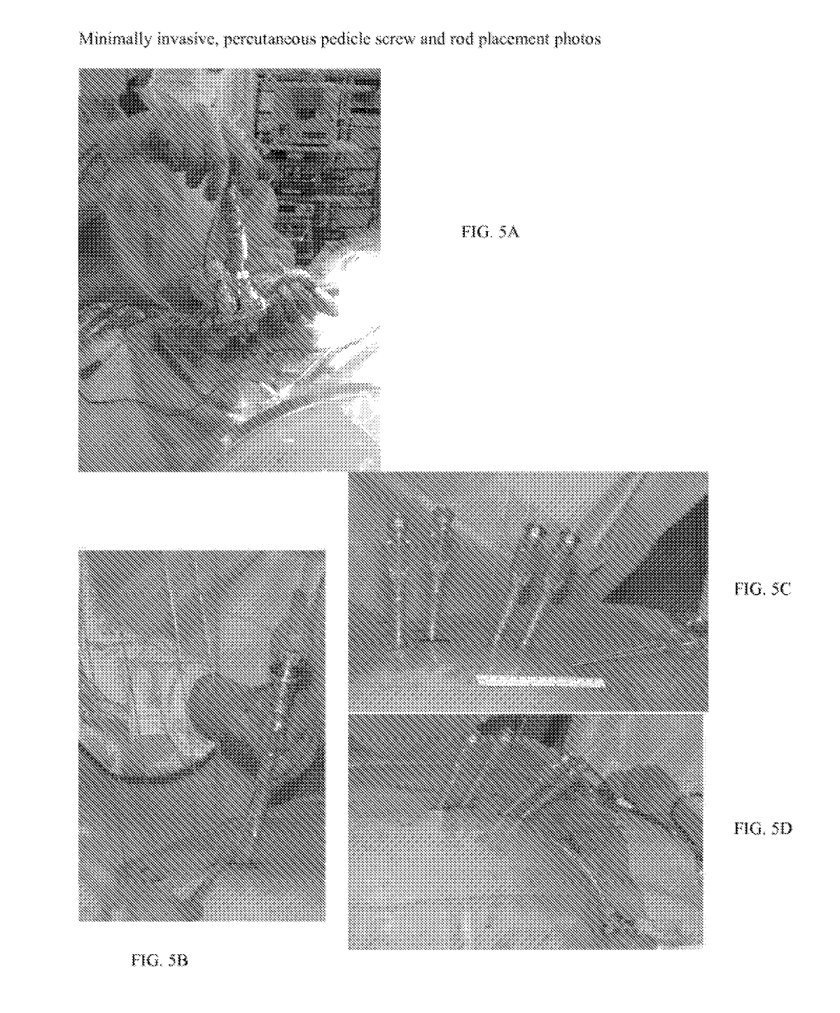

The advent of minimally invasive spine surgery has led to unique approaches to placing spinal fixation, as well as performing spinal arthrodesis. Notably, ALIFs can now be performed via a lateral transpsoas approach as seen in FIGS. 3A-3D (lateral lumbar interbody fusion or LLIF, which has replaced the use of standard anterior retroperitoneal approaches for many surgeries. Additionally, minimally invasive transforaminal lumbar interbody fusions (TLIF) illustrated in FIG. 4, which are performed through modification of the posterior approach to the spine has been popularized over the past decade, as well. The use of such interbody fusion techniques in the lumbar spine provides a stronger potential for fusion completion over posterior fusion alone, yet a combination of anterior and posterior lumbar fusion techniques results in the highest probability of fusion completion.

Performing standard open posterior spinal exposures, fusions, and fixation carries a considerably high risk of infection, muscle damage, longer hospital stays, and greater blood loss than with less invasive techniques, such as percutaneous methods of introducing posterior spinal fixation. The most common method of stabilizing the spine is through transpedicular screw and rod fixation, and it can be performed through percutaneous and other less invasive means.

Importantly, percutaneous means for introducing posterior spinal pedicle screw fixation is performed through small incisions without direct visualization as illustrated in FIGS. 5A-5D, and through the use of fluoroscopic or navigation guidance. The small incisions, and quite often, deep wounds result in an inability to adequately perform the posterior spinal arthrodesis/fusion as part of the procedure. In order to perform a posterior fusion concurrently, one must enlarge the exposure or incisions, which can result in greater tissue damage. In some cases, the exposure is simply not feasible due to depth or challenged tissue retraction. These challenges also result in a considerable increase in surgery time if one chooses to undertake the posterior fusion.

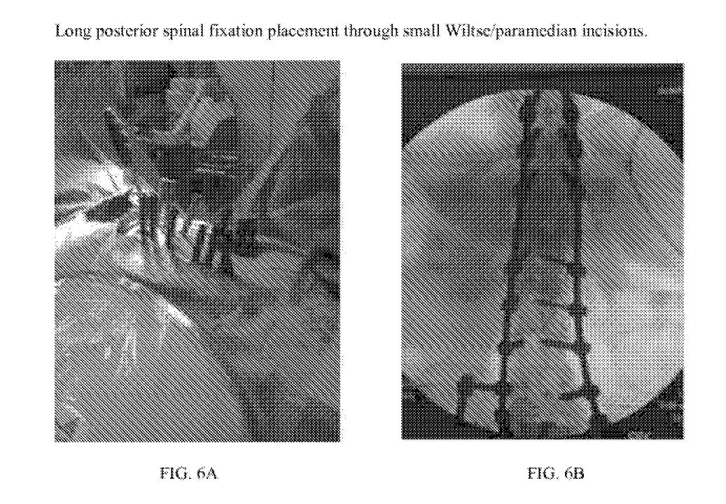

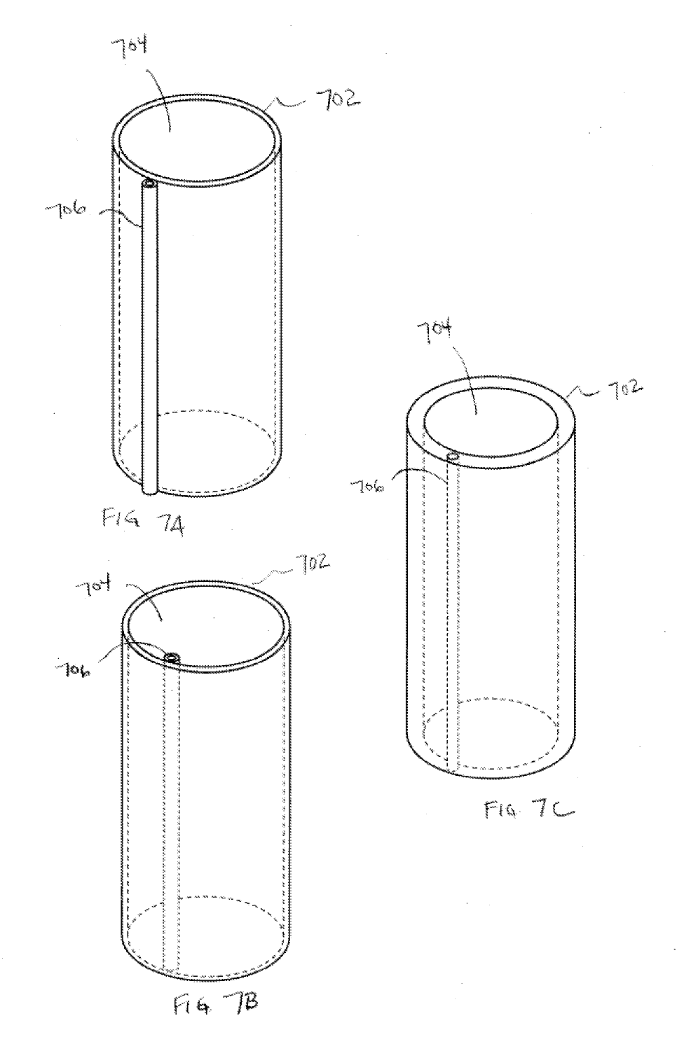

Because minimally invasive techniques for transpedicular spinal fixation have become increasingly common as augments to anterior, lateral, posterior, and transforaminal lumbar interbody fusions, there is a growing need to provide acceptably efficient and focused means of performing supplementary posterior facet fusions during the process of transpedicular screw fixation. The capacity for modern minimally invasive fixation systems to be placed over very long segments of the thoracolumbar spine as seen in FIGS. 6A-6B, including for scoliosis correction, supports the need for such minimally invasive fusion visualization techniques.

Given the background information and current trends, a tubular retractor and visualization system can facilitate spinal fusion procedures. The system incorporates several key features in allowing for maximally efficient posterior spinal facet fusions during minimally invasive transpedicular spinal fixation placement.

In an exemplary embodiment, the tubular retractor docks on the posterior spine through a small 16-30 mm paramedian skin or fascial incision via a transpedicular wire or pedicle screw head/tower reference point. By using the guide wire within the pedicle, or the transpedicular screw as eccentric reference points, the tubular retractor can be rotated into medialized position for retraction of the multifidi/paraspinal muscle mass that overlies the facet joint. The retractor can be stabilized, if necessary, via table-mounted rigid arm, and lighting can be provided by external source, such as a headlight or microscope, versus internal lighting capacity via light source.

Rotational placement of the tubular retractor relative to the transpedicular reference point also allows for lateral positioning over the transverse processes at both the caudal and cephalad levels, which provides for access to completing an intertransverse posterolateral fusion, if so desired.

The tubular retractors can have a circular, ovoid, or other shape, and can be designed with flat and oblique deep surfaces to match the posterior spinal column morphology at the location of docking.

Additionally, currently, posterior spinal fixation is not a reimbursable code without the performance of a posterior or posterolateral arthrodesis. Supplemental posterior fixation after ALIF and LLIF is not reimbursable based on utilization of the codes for these particular procedures, so one must perform a separate and distinct posterior arthrodesis (facet or intertransverse fusion) concurrently with transpedicular, interspinous, or facet fixation, in order to code for posterior fixation and to comply with current coding rules.

This coding situation results in a distinct incentive and need beyond clinical merit to incorporate a concurrent posterior fusion in one's plan to perform a posterior spinal fixation.

Retractors

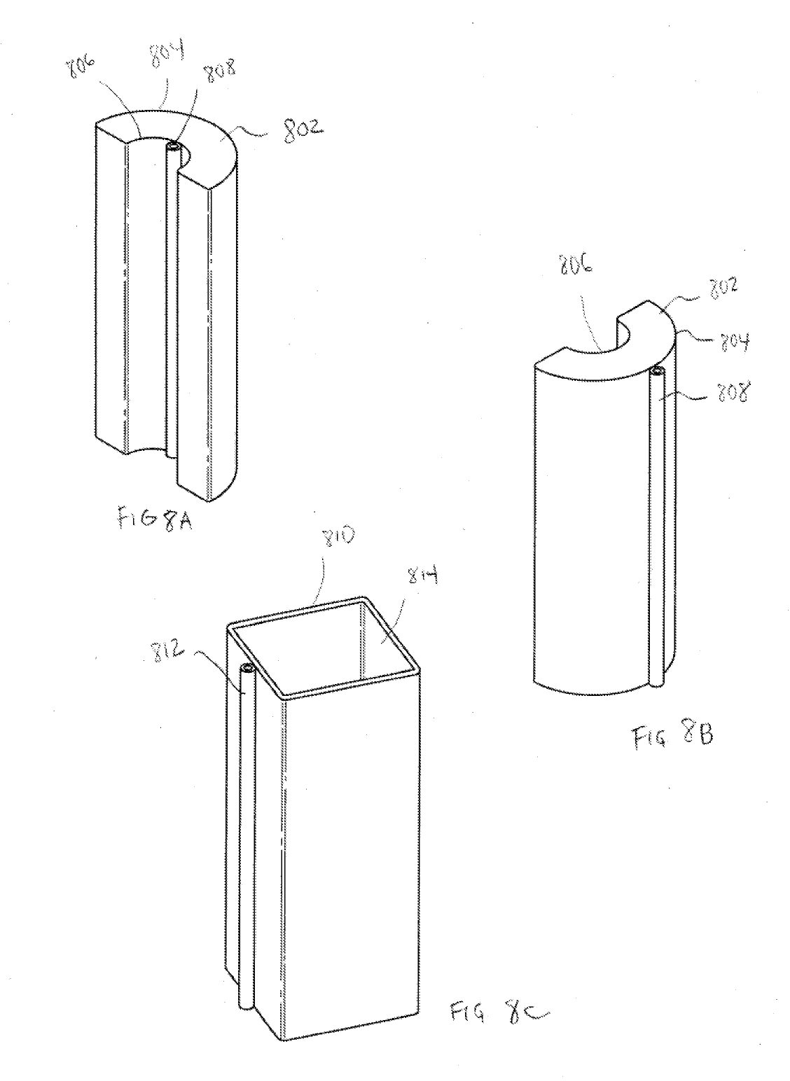

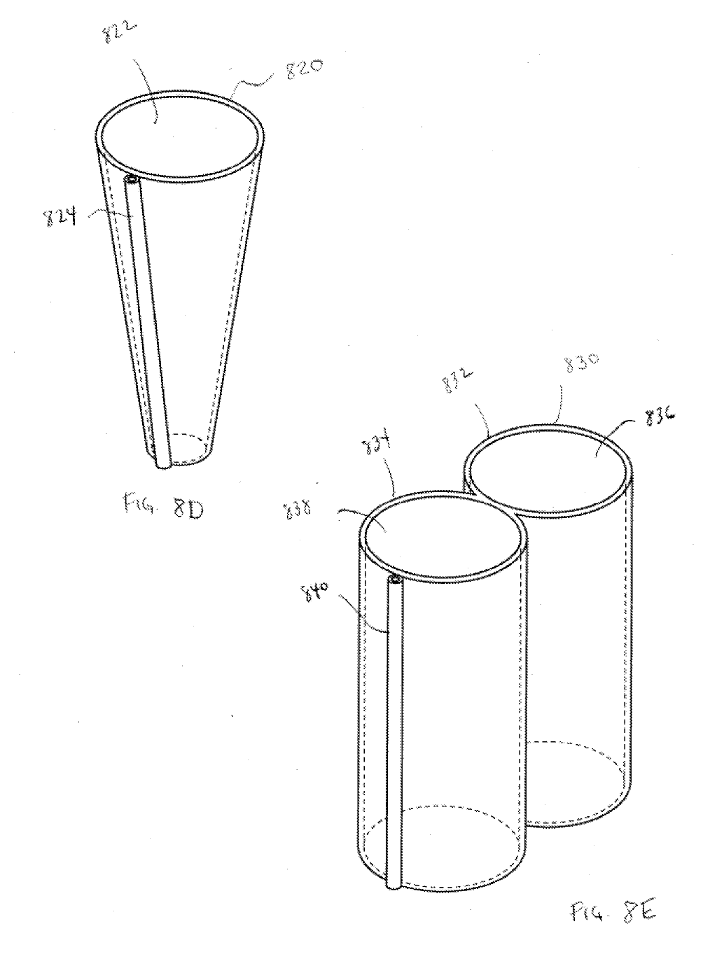

Retractors which may be used to perform the surgical procedure described above as well as other surgical procedures are disclosed herein. FIGS. 7A-7C illustrate several variations of an embodiment of a tubular retractor. In FIG. 7A a tubular retractor 702 is cylindrically shaped and has a central bore 704 extending through the tubular retractor and the central bore is sized to receive surgical instruments and allow a surgical access to the surgical field created by the walls of the tubular retractor retracting tissue. Additionally, a channel 706 is disposed along the outer surface of the tubular retractor. The channel is sized and adapted to receive an anchoring element such as a guidewire or a pedicle screw tower so that the retractor is then anchored to the anchoring element and positionable therearound. In FIG. 7A the channel is on an outer surface of the tubular retractor, but in FIG. 7B the channel is on an inner surface of the tubular retractor and in FIG. 7C the channel is within the wall thickness of the tubular retractor.

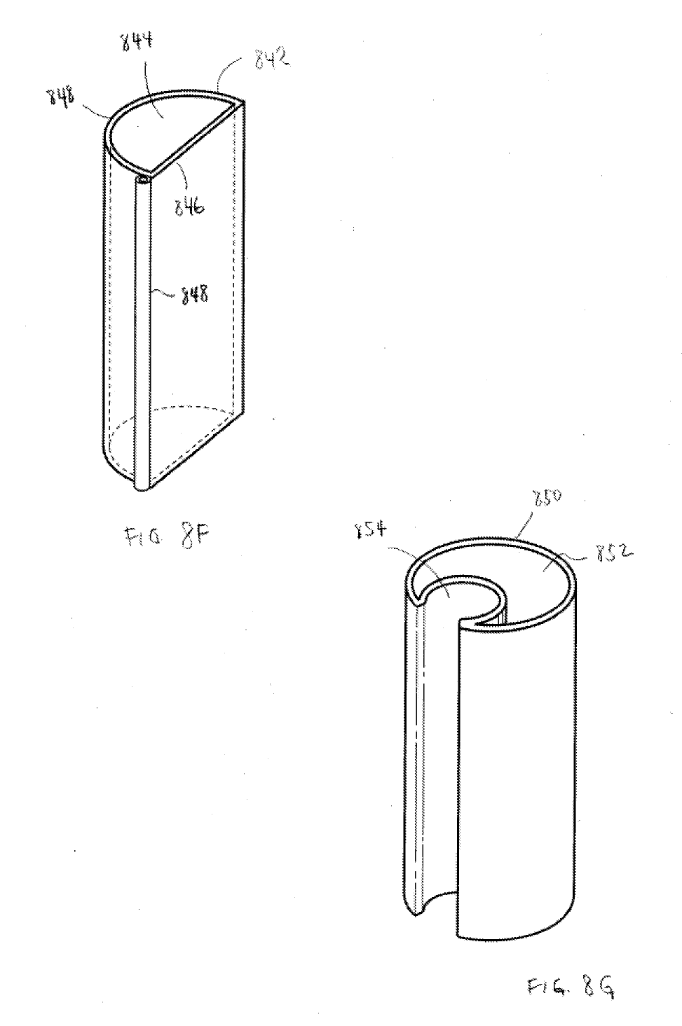

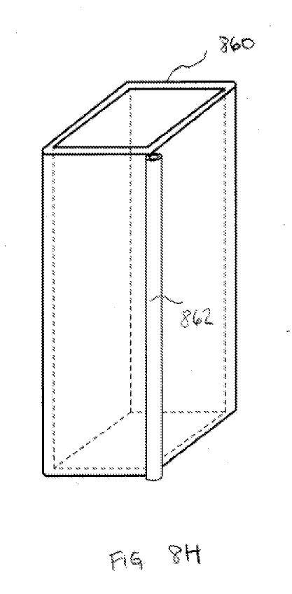

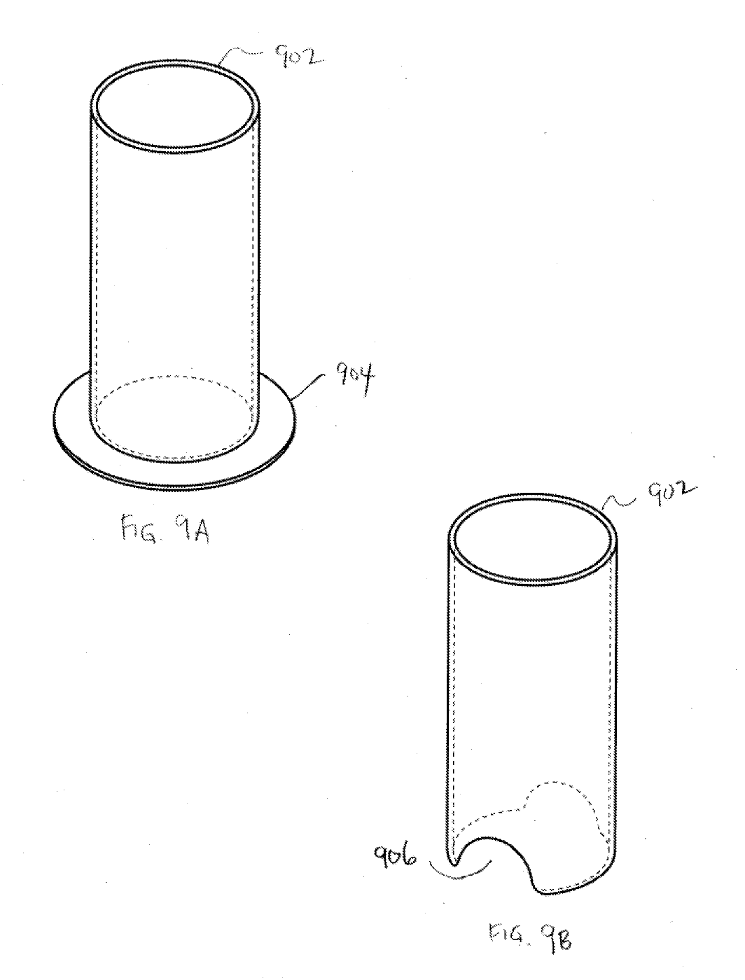

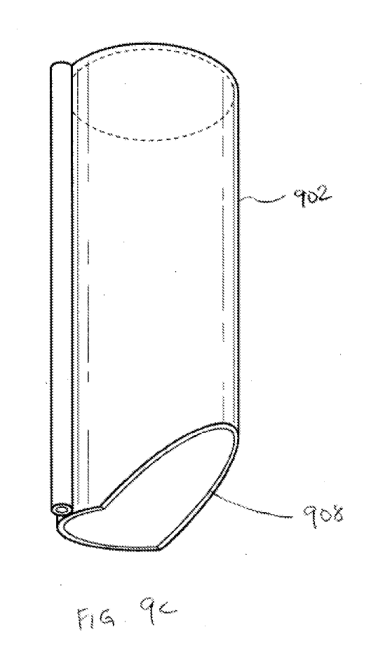

The retractor is not limited to being a cylinder. FIGS. 8A-8G illustrate alternative embodiments. FIG. 8A illustrates a C-shaped or hemicylindrical shaped retractor 802 having a convex surface 804 and a concave surface 806. Additionally, channel 808 for receiving the anchoring element is disposed on the concave portion of the retractor. FIG. 8B illustrates a similar embodiment with respect to FIG. 8A, with the major difference being that the channel 808 is now on the convex part of the retractor. FIG. 8C illustrates another embodiment where the retractor is a square or rectangular shape 810 having a corresponding square or rectangular bore 814 extending therethrough and the channel 812 may be on an outer surface of the retractor as seen, or it may be on the inner surface of the retractor or in the wall of the retractor as previously described.

FIG. 8D illustrates another embodiment of a tapered tubular retractor 820. The bore 822 is also tapered and the channel 824 may be on the outer surface as seen, or on the inner surface or in the wall of the retractor 820. FIG. 8E illustrates a retractor 830 having two cylindrical portions 832, 834 coupled together to form a figure eight pattern. Each cylinder has a bore 836, 838 and the channel 840 for receiving the anchoring element may be on the outer surface as seen, or it may be on an inner surface of either retractor or disposed in the wall of either retractor. The two cylinders may be releasably coupled to one another or they may be integrally formed together.

FIG. 8F illustrates a tubular retractor 842 having a D-shaped tube with bore 844 and convex outer surface 848 and flat outer surface 846. The channel 848 is shown on a corner of the retractor and on the outer surface, but one of skill in the art will appreciate that the channel may be positioned anywhere along the outer surface of the retractor, or anywhere along the inner surface or in the wall of the retractor. In this or any of the embodiments described herein, the channel may be integrally formed with the retractor or it may be a discrete component fixedly or releasably attached to the retractor. The channel is generally designed to be loaded over a guidewire and then slid down into the surgical field. However, in other embodiments, the channel may be snapped or pressed into the anchor element as seen in FIG. 8G. The partial tubular retractor 850 includes a partial tube as the retractor blade is formed into a crescent like shape and a second partial tube or crescent shape 854 is formed into the retractor. The second partial tube may be slid over an anchoring element, or it may be laterally snapped or loaded into engagement with the anchoring element. The bore of the retractor is also crescent shaped 852. FIG. 8H illustrates still another embodiment where the retractor 860 has a diamond shaped cross-section with the channel 862 preferably coupled to the outer surface of the retractor and preferably adjacent a corner, although it may be on an inner surface or in the wall of the retractor and anywhere along the perimeter.

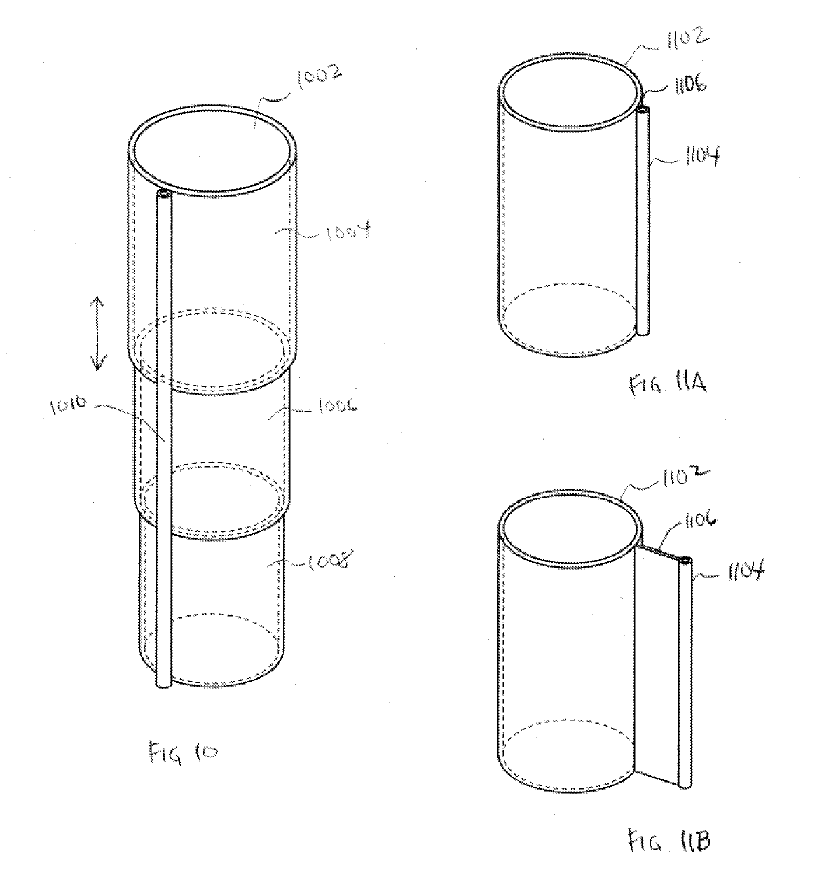

FIG. 9A illustrates another feature which may be used with any of the retractors disclosed herein. As will be described below, the tubular retractor is used to retract tissue away from the bore. In some cases, the retractor will be positioned or rotated to push additional tissue out of the way. This tissue will tend to roll off the distal end of the tubular retractor and then re-occupy and obstruct the surgical field. Thus a flange 904 near the distal end of the retractor is useful for preventing tissue from sliding off the tip of the retractor. FIG. 9B illustrates another feature which may be used alone or in combination with any of the retractors described herein. When the retractor is rotated about the anchoring element such as a guidewire or pedicle screw tower, the bottom of the tube will sweep in an arc over the tissue in the surgical field. However, if the anatomy is not flat, the bottom of the retractor may bump into raised areas in the surgical field such as a bone or other protruding object. Thus, the distal end of the retractor may be contoured 906 to have recessed areas that accommodate raised areas, and thus the retractor may be rotated over the raised areas without interfering with rotation of the retractor. Similarly, the distal end of the retractor 902 may be beveled 908 as seen in FIG. 9C. Preferably the bevel is disposed on a leading edge of the retractor as it rotates.

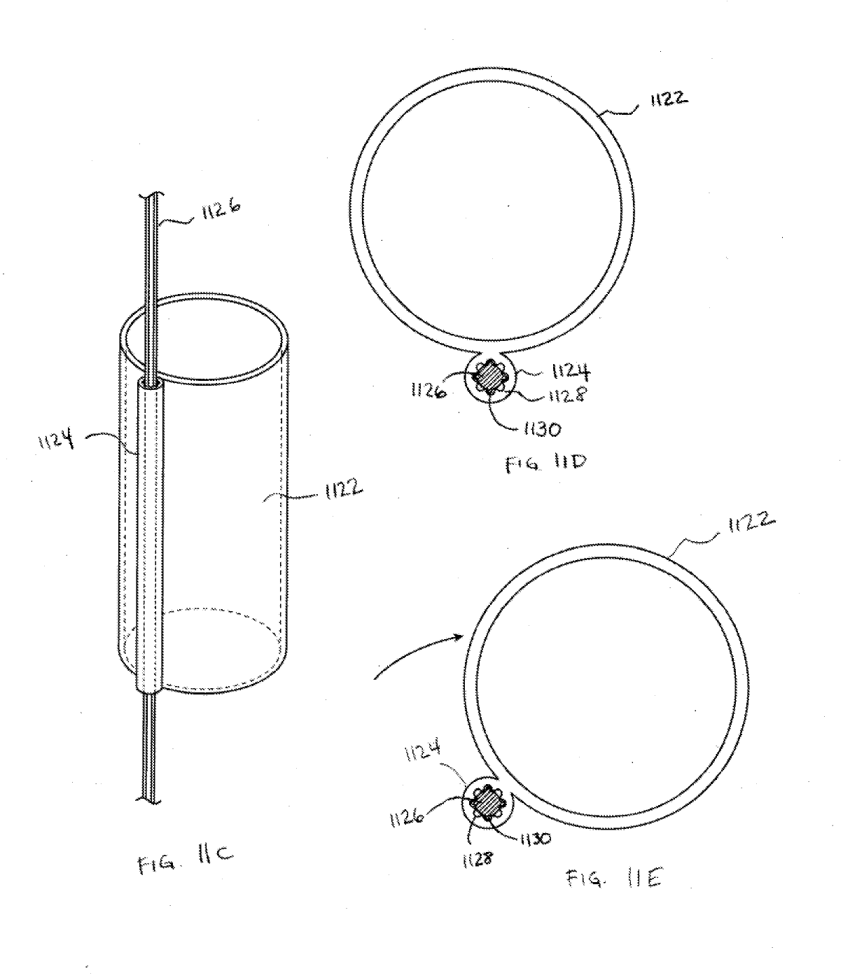

In some situations, it would be desirable to provide a retractor having an adjustable length. FIG. 10 illustrates an exemplary embodiment of a retractor 1002 having a plurality of tubular bodies 1004, 1006, 1008 stacked within one another to create a telescoping retractor. Thus, the retractor length may be adjusted as needed. A locking mechanism (not illustrated) will hold the retractor in the desired length. Exemplary locking mechanisms include ratchet mechanism, detents, collets, etc. Additionally, the channel 1010 may also be telescopically adjustable to match the length of the retractor, and the channel may be on the inner or outer surfaces of the retractor or in the wall as described above.

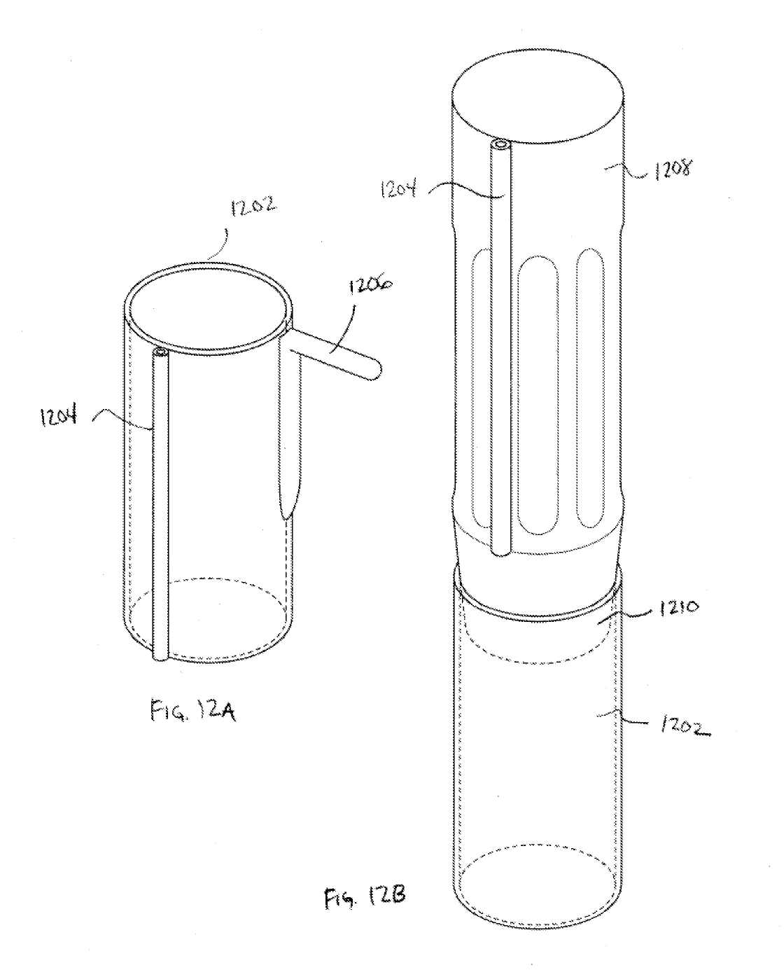

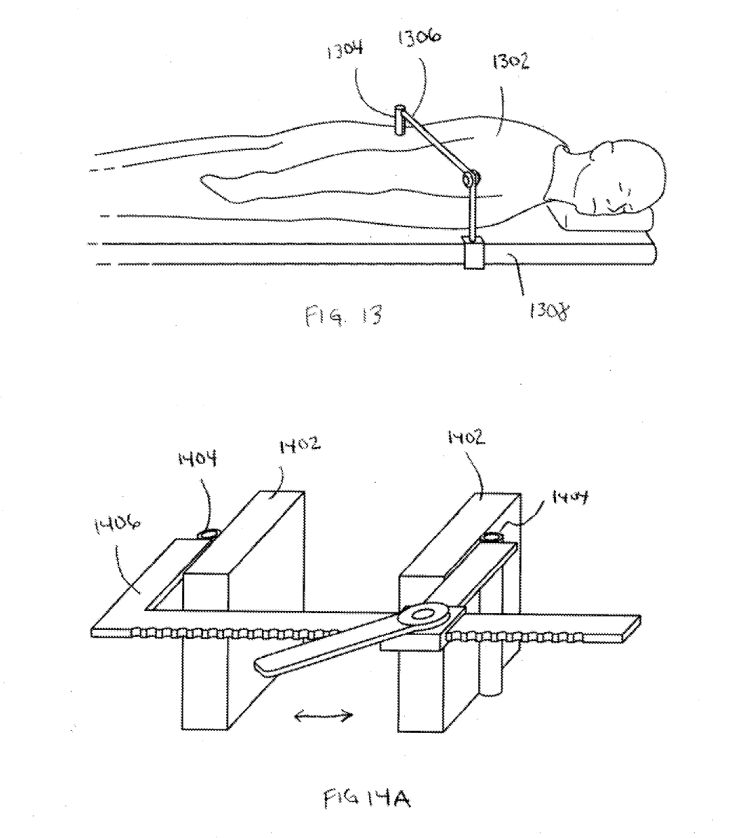

FIGS. 11A-11B illustrate an embodiment of a retractor 1102 having a laterally adjustable channel 1104. In FIG. 11A the channel 1104 is positioned adjacent to the outer surface of the retractor 1102. In FIG. 11B, the channel has been displaced laterally away from the outer surface of the retractor by a distance 1106. Thus, in FIG. 11B when the retractor is coupled to the anchoring element via channel 1104, it can be rotated with a larger sweep than compared with FIG. 11A. The sweep adjustment in FIGS. 11A-11B may also be combined with the length adjustment feature illustrated in FIG. 10. The channel 1104 may be adjustably coupled to the retractor 1102, or it may be removable and thus various size attachments may be attached to the retractor for controlling the rotatation sweep, or an adjustable element coupled with the retractor may be actuated to adjust the rotation sweep.

In some embodiments is may be advantageous to provide a mechanism for controlling rotation of the retractor around the anchoring element. FIGS. 11C-11E illustrate one exemplary embodiment that provides a surgeon with feedback on rotation. In FIG. 11C a tubular retractor 1122 with channel 1124 is disposed over the anchoring element 1126, here a guidewire or pedicle screw post. FIG. 11D illustrates a top view of FIG. 11C and FIG. 11D shows rotation of the retractor about the guidewire. The channel 1124 includes a detent 1130 with longitudinally oriented cutouts or notches that extend radially inward into the wall surrounding the channel 1124, and the anchoring element 1126 includes one or more evenly spaced ball detents or other protuberances 1128 disposed around the circumference. The notches may be spaced apart with any desired interval, for example notches may be spaced apart every ninety degrees such that as the retractor is rotated about the anchoring element, the detent will engage a notch every quarter turn. The surgeon may feel or hear the clicking and thus tactile as well as auditory and visual feedback are provided. The mechanism may also be used to prevent anti-rotation once the surgeon has rotated the retractor into a desired position.

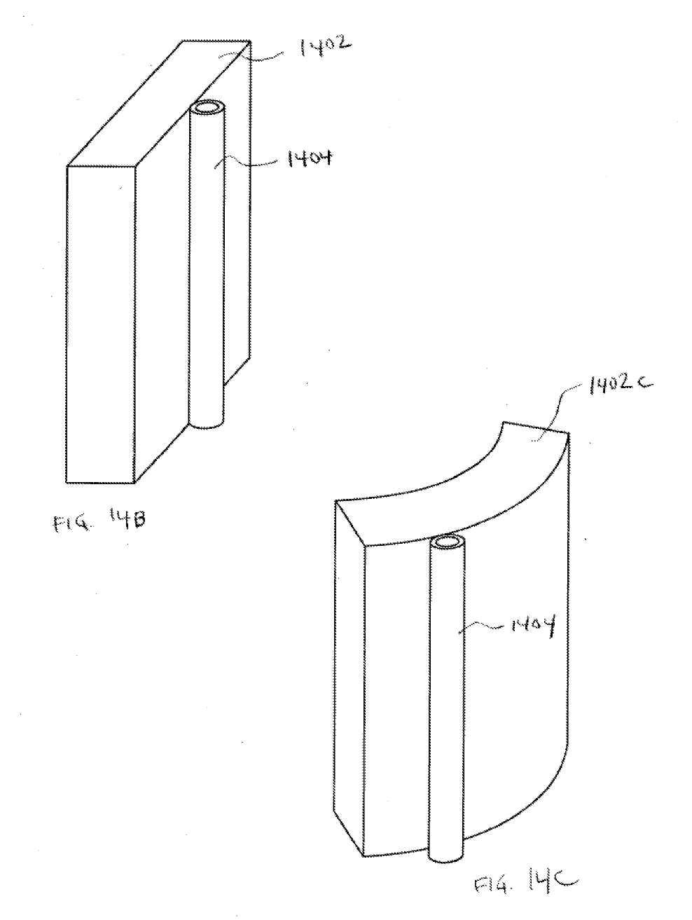

FIGS. 12A-12B illustrate embodiments of tubular retractors with handles for actuating the retractor. FIG. 12A shows a handle 1206 having an arm extending radially outward that allows the retractor 1202 to easily be rotatated about channel 1204 when engaged with an anchoring element. The handle 1206 may be releasably attached to the retractor or it may be permanently attached thereto. In FIG. 12B a tubular handle 1208 is disposed in the bore of the retractor 1202 and a tapered distal portion 1210 of the handle frictionally engages the two components together. The handle may extend partially or entirely through the bore of the retractor. The tubular handle may be actuated in any direction to move or rotate the retractor. The tubular handle may also have a central bore extending therethrough in order to allow a surgeon to see through the bore of the retractor and/or to allow instruments to be positioned in the bore. The tubular handle may be removed once the retractor has been positioned. The channel 1204 for receiving the anchoring element may be disposed on the handle or on the retractor, or on both portions.

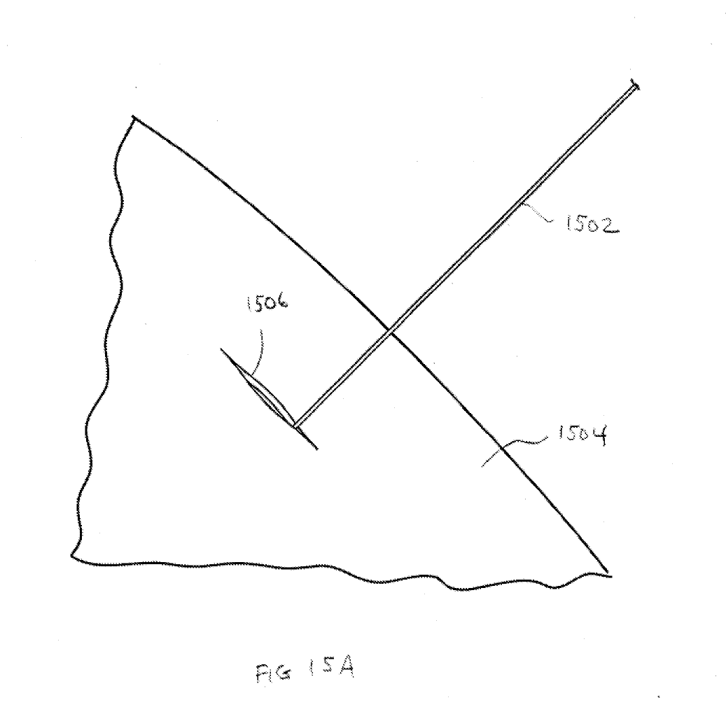

FIG. 13 illustrates a patient 1302 lying down on a surgical table 1308. The retractor 1304 has been placed in the patient and an optional arm 1306 may be used to hold the retractor in position. The arm may have an adjustable end and the opposite end may be fixed to the table.

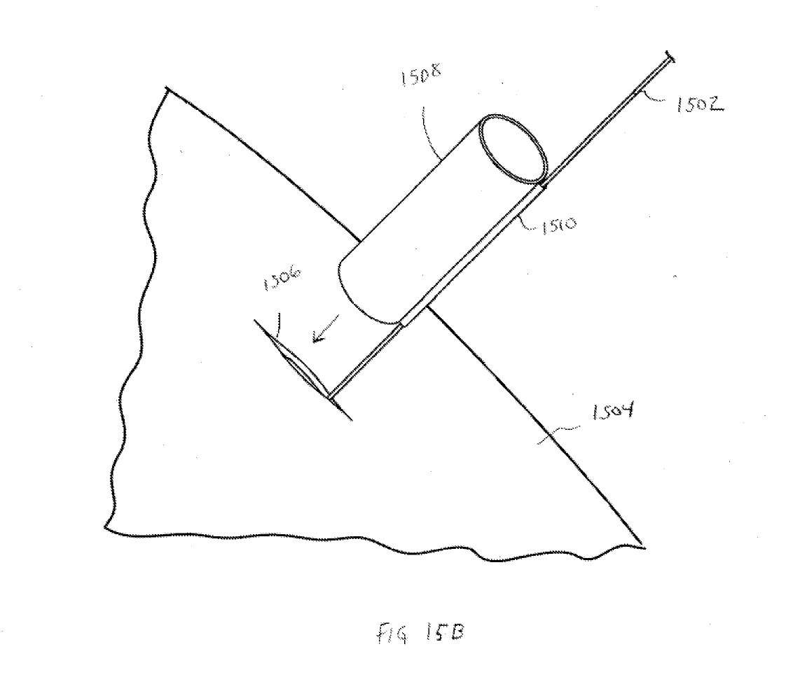

FIG. 14A illustrates still another embodiment of a positionable retractor. Retractor blades 1402 may be be releasably coupled to a frame 1406 that can be opened and closed to adjust the amount of spreading between the retractor blades 1402. Optionally, a channel 1404 may be coupled to either retractor blade or ensuring proper location or for serving as an anchor point. Thus the retractor blade or blades may be advanced over an anchoring element such as a guidewire or pedicle screw tower. The frame may then be attached to the retractor blades to spread the blades apart. FIGS. 14B-14C illustrate exemplary blade shapes that may be used with the frame 1406 in FIG. 14A. For example, the blade may be rectangular 1402, or the blade may be curved 1402C. Other shapes may be used depending on the anatomy being treated and the surgeon's preference.

Any of the retractors may include radiopaque markers so that they can be easily observed with radiography. Also, any of the retractors may also be radiolucent so that the retractor does not obstruct observation of surrounding tissue.

Illumination

Any of the retractors described herein may also be used to illuminate the surgical field. A separate illumination system such as fiber optic cables may be coupled to the retractors to deliver light to the surgical field, or in preferred embodiments, the retractor itself includes a non-fiber optic waveguide or is a waveguide to transmit light from a source through the retractor by total internal reflection and then the light is extracted and directed to the surgical field. U.S. patent application Ser. No. 11/397,446 (now U.S. Pat. No. 7,510,524); Ser. No. 11/715,247 (now U.S. Pat. No. 7,901,353); Ser. Nos. 13/429,700; 12/188,055; 12/412,764 (now U.S. Pat. No. 8,162,824); Ser. Nos. 13/019,198; 12/191,164; and 13/026,910 describe other aspects of illuminated retractors which may be used in conjunction with any of the retractors described herein; the entire contents of each are incorporated herein by reference. The waveguide may be injection molded and thus the waveguide may be a single homogeneous material.

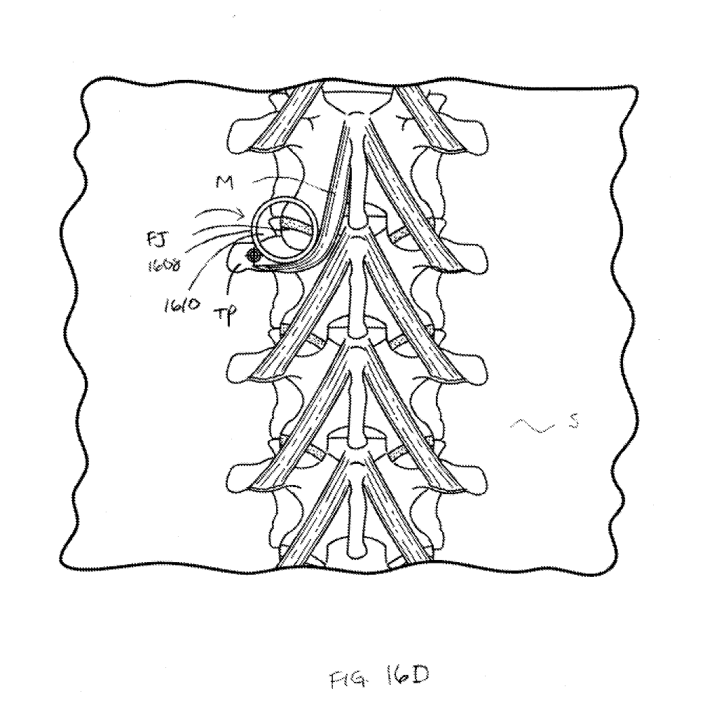

FIG. 17 illustrates a side view of a COP illuminating waveguide 17250 with a proximal end 17251 and a distal end 17252 that is inserted into a patient's via an incision. The waveguide 17253 may also be used as a general speculum, retractor or anoscope and is preferably formed of an optically efficient polymer such as polycarbonate, cyclo olefin polymer (COP) or cyclo olefin copolymer (COC). It may also include an input connector 17254 that serves to conduct light into the waveguide such that light is conducted around the entire circumference 18255 of the waveguide tube. Output optical structures 17256 are typically placed near the distal end on the inside wall 17257 along all or a portion of circumference 18255. Output optical structures placed on the end face 17258 or outside wall 17259 might cause irritation to the cavity walls during insertion. If output optical structures are required on end face 17258 or outside wall 17259, any suitable coating or material may be used to lessen the irritation to the patient's body tissue during insertion of the waveguide. The output optical structures provide even illumination of the entire cavity wall. A reflective or prismatic surface may also be created on the proximal end face to send mis-reflected light rays back toward the distal output optical structures. In this embodiment or any of the embodiments disclosed herein, the features used to extract light from the waveguide may be disposed on an inner surface of the waveguide, or an outer surface of the waveguide, or they may be disposed on both surfaces. Additionally, the extraction features may be disposed anywhere along the waveguide, including the distal face, the distal portion, a proximal portion, or a region in between the proximal and distal portions of the waveguide. The extraction features may also be disposed on more than one region of the waveguide, and the extraction features are not limited to those described in this specification.

Referring now to FIG. 18 shows an example of a light directing structure that contributes to light distribution around circumference 18255. Light entering input connector 17254 may be directed by a light control structure, such as structure 18260, which splits the incoming light and sends it down into the waveguide tube wall at an angle ensuring circumferential light distribution.

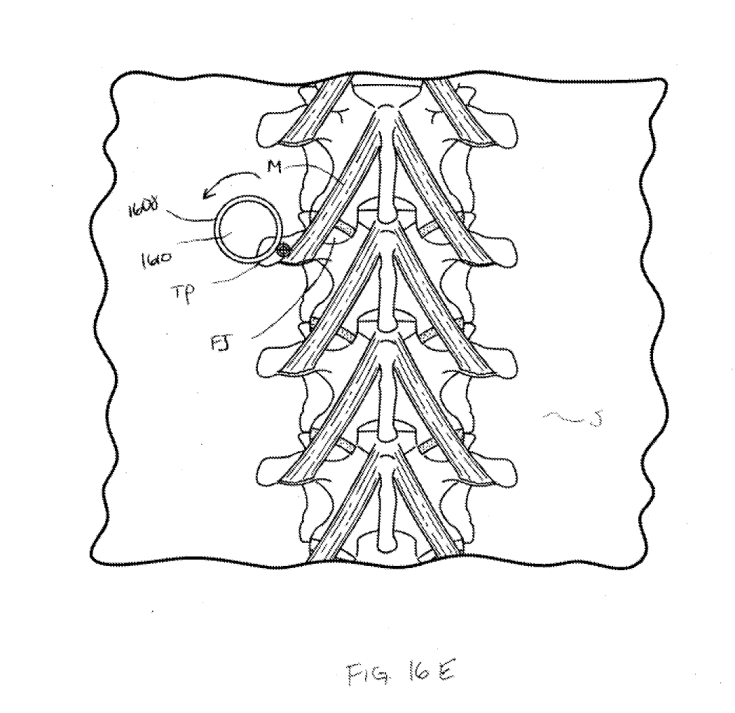

Referring now to FIG. 19, optical waveguide 19270 may include an alternate light coupling apparatus such as coupling 19271. Coupling 19271 may provide mechanical support and optical conduit between optical input 19272 and waveguide 19270.

Any of the optical waveguides may include a pigtail fiber for inputting light into the waveguide. The pigtail fiber is one or more optical fibers having one end integrally connected to the waveguide. This may be fabricated by insert molding or co-molding the waveguide over the optical fibers. In alternative embodiments one end of the pig tail may be adhesively coupled to the waveguide. The opposite end of the pigtail may have a connector for optically coupling with a light source. Still other embodiments may have a plurality of pigtails for coupling the waveguide with one or more optical sources.

Additionally, any of the embodiments described herein may include a smoke evacuation feature. A channel may be formed in the waveguide or the retractor, or a tube may be coupled thereto and vacuum applied to evacuate smoke or other undesirable fumes from the surgical field. Additionally, various markers may be coupled to the retractor to enable visualization and help with positioning of the retractor. For example, radiopaque markers may be added to allow visualization under fluoroscopy. Metalized coatings may also be used to help with positioning by allowing tracking of the retractor. Magnetic field markers may be placed on the retractor to allow tracking of the device. The metalized coating allows the magnetic markers to be coupled to the retractor. In still other embodiments, the retractor may be radiolucent during fluoroscopy or other imaging techniques in order to allow a surgeon to visualize the tissue without interference from the retractor.

Distal end 17276 as shown in IG. 20 includes one of more vertical facets such as facet 20276F within the distal end to disrupt the light spiraling within the waveguide. Also shown are structures such as structure 20278 on the end face of the cannula which serve to direct light as it exits the end face. Shown are convex lenses, but concave lenses or other optical structures (e.g., stamped foil diffuser) may be employed depending on the desired light control. Stepped facets such as facets 20279 and 20281 are shown on the outside tube wall. The “riser” section, risers 20279R and 20281R respectively, of the stepped facet is angled to cause the light to exit and as a result the waveguide slides against tissue without damaging the tissue. The angle is generally obtuse relative to the adjacent distal surface. Steps may be uniform or non-uniform as shown (second step from end is smaller than the first or third step) depending on the light directional control desired. The steps may be designed to direct light substantially inwards and or toward the bottom of the tube or some distance from the bottom of the tube, or they may be designed to direct light toward the outside of the tube, or any suitable combination. The facets may be each designed to direct light at different angles away from the waveguide and or may be designed to provide different beam spreads from each facet, e.g., by using different micro-structure diffusers on each facet face.

Facets may be used on the inside surface of the COP waveguide, but if waveguide material is removed to form the facets, the shape of the waveguide may be changed to maintain the internal diameter of the bore generally constant to prevent formation of a gap between the waveguide and a dilator tube used to insert the waveguide into the body. Said gap may trap tissue, thereby damaging it during insertion into the body or causing the waveguide to be difficult to insert. Thus the outer wall of the waveguide may appear to narrow to close this gap and prevent the problems noted.

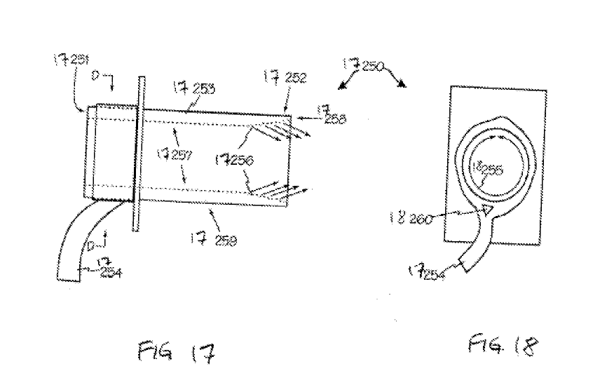

Referring now to FIGS. 21-23, applied light energy 21282 may be bifurcated to send light into wall 22284 of COP waveguide or tube 21286. Light input 21288 may be split in input coupling 21290.

The bifurcated ends 21290A and 21290B of input 21288 preferably enter tube wall 22284 at an angle 22291 to start directing light around the tube wall. Alternatively, the bifurcated ends 21290A and 21290B may each enter tube wall 22284 at different angles to further control light distribution. The bifurcated ends may enter the tube wall orthogonally, but this may require a prism structure in the wall placed between the input and the output with the apex of the prism pointed at the input. The prism structure directs the light around the tube wall. A vertical prism structure, prism 21292 is shown with apex 21292A of the prism pointed in toward the center of the tube. Prism structure 21292 may direct a portion of the input light back underneath the inputs and contributes to directing light all the way around the tube wall. The position, angle and size of this prism relative to the input bifurcated end determines how much light continues in the tube wall in its primary direction and how much light is reflected in the opposite direction in the tube wall. In other embodiments, the light input may be trifurcated or split into any number of light input arms or fiber optics. Additionally, other optical microstructures may be used to control the light rather than relying on just prisms.

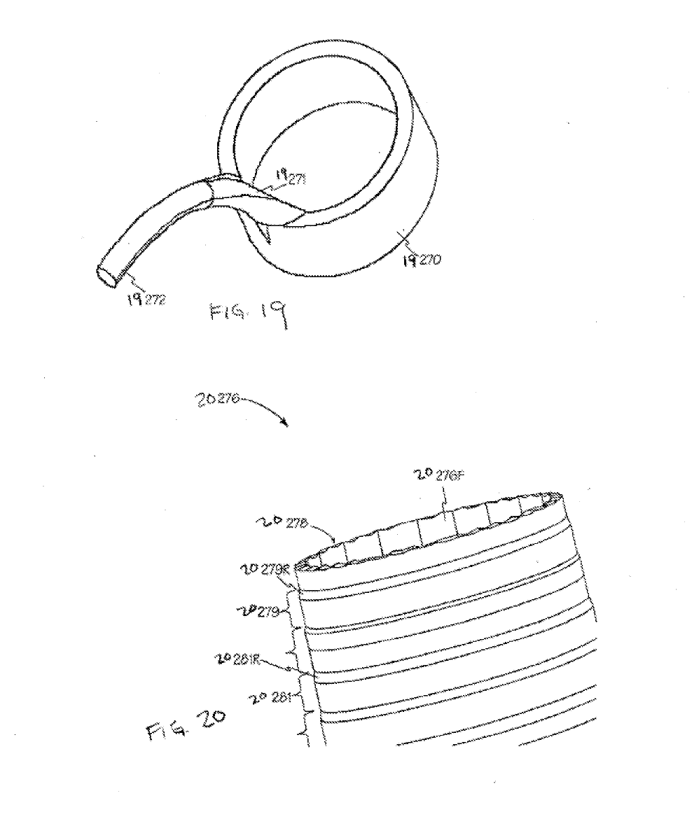

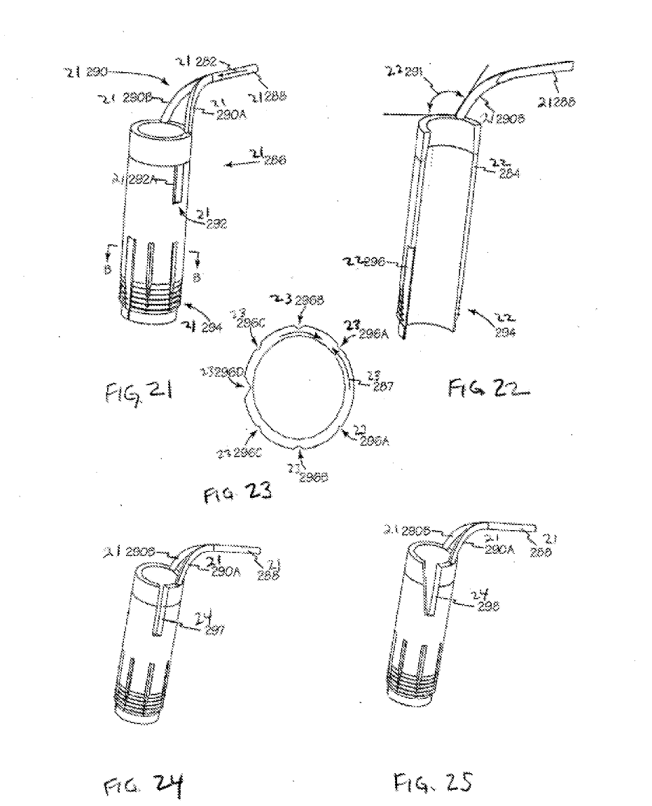

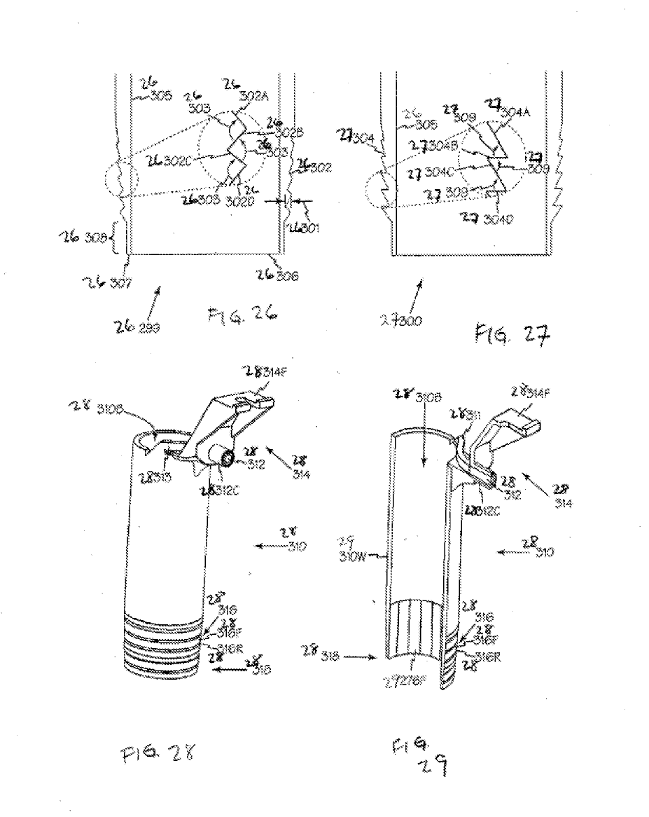

Additional vertical prism structures or light disruption structures may be placed toward the bottom of the tube on the outside tube wall as shown in FIGS. 21-23. One or more light extraction structures 21294, shown as circumferential grooves cut into the outside wall of the tube, may also be included to optimize the illumination provided below waveguide 21286. Light 23287 traveling circumferentially in the tube wall will not strike the light extraction structures 21294 with sufficient angle to exit waveguide 21286. Thus, vertical prism 22296 or light disruption structures such as disruption prisms 23296A, 23296B, 23296C and 23296D may be necessary to redirect the light so that the light rays 23287 will strike light extraction structures 21294 and exit the tube wall to provide illumination. As shown in FIG. 23, vertical prism structures such as 23296A and 23296B have different depths around the circumference in order to affect substantially all of the light rays traveling circumferentially in the tube wall. Vertical prisms of constant depth would not affect substantially all of the light rays.

FIG. 22 also illustrates how a COP half-tube may be formed to provide illumination. At least one COP half-tube illuminator may be attached to the end of at least one arm of a frame, such as that used in Adson, Williams or McCulloch retractors. Such frames typically include two arms, but some frames have more than two arms. The arms of the frame are then moved apart to create a surgical workspace, with the at least one half-tube illuminator providing illumination of said space. One or more half-tube illuminators may also be provided with an extension that preferably is in contact with the opposite half tube and that serves to prevent tissue from filling in the gap created when the half tubes are separated. Tissue may enter this gap and interfere with surgery, so the extension helps reduce that issue.

FIGS. 24-25 illustrate alternative configurations of an illumination waveguide. Proximal reflecting structures such as proximal structure 24297 and proximal structure 24298 may provide more complete control of the light within the waveguide with an associated weakening of the structure.

Referring now to FIGS. 26-27, cross-sections 26299 and 27300 illustrate additional alternate light extraction structures of the distal end of an illumination waveguide. As shown with respect to FIG. 20 above, depth 26301 of light extraction structures such as structures 26302 and 27304 increases relative to the distance from the light input in order to extract most of the light and send the light out the inner tube wall 26305 toward the bottom or distal end 26306 of the tube. The light that remains in the tube wall below the extraction structures exits the bottom edge 26307, which may be flat or may have additional optical structures, e.g., a curved lens or a pattern of light diffusing structures such as structures 20278 of FIG. 20. In FIG. 26, the distal 5-10 mm of the tube wall, window 26308, have no structures to enable this surface to operate as a window to the surrounding tissues to improve visualization of the surgical space. As illustrated in FIG. 26, light extraction structures 26302 are formed of adjacent facets such as facets 26302A, 26302B, 26302C and 26302D forming angles 26303 between adjacent facets. In this illustration angles 26303 are obtuse.

As illustrated in FIG. 27, light extraction structures 27304 are formed of adjacent facets such as facets 27304A, 27304B, 27304C and 27304D forming angles 27309 between adjacent facets. In this illustration angles 27309 are acute. Any suitable angle may be used.

It has been demonstrated that a clear waveguide cannula provides improved visualization of the entire surgical workspace because the surgeon can see the layers of tissue through the walls, thereby enhancing the surgeon's sense of depth and position, which are difficult to determine in an opaque cannula. Light exiting the side walls at the areas of tissue contact, due to changes in total internal reflection at these contact areas, serves to illuminate these tissues making them more visible than if a non-illuminated, non-waveguide clear plastic cannula is used. Alternatively, extraction structures 302 or 304 may extend all the way down to bottom edge 26307.

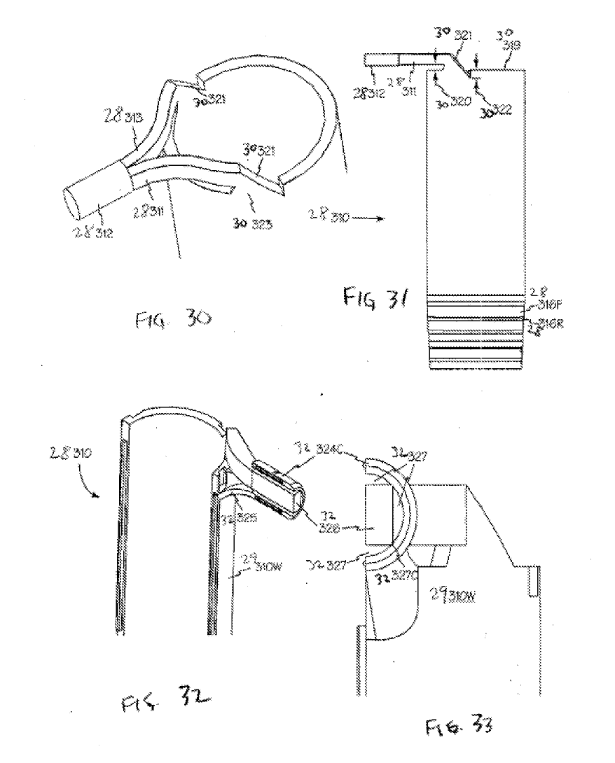

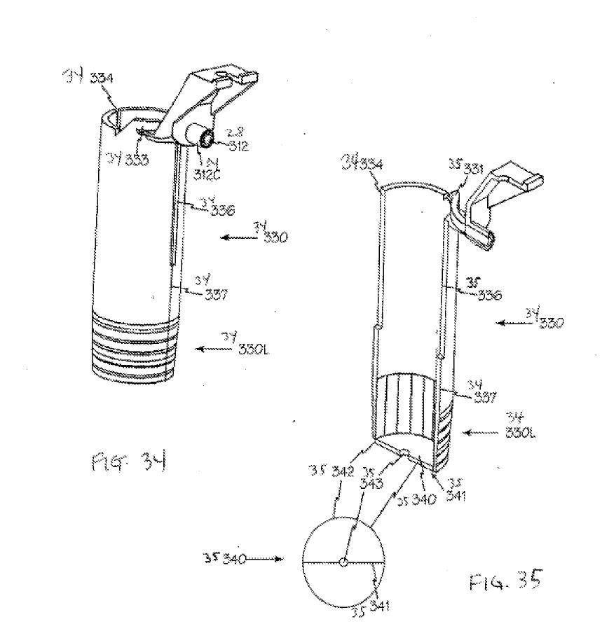

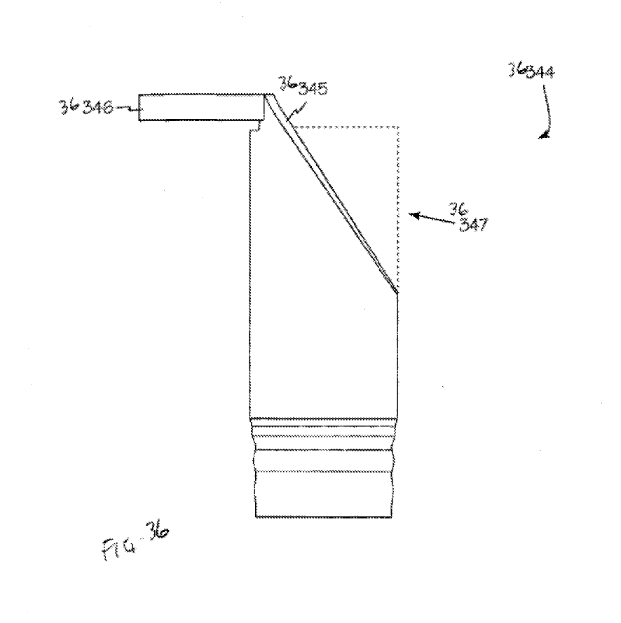

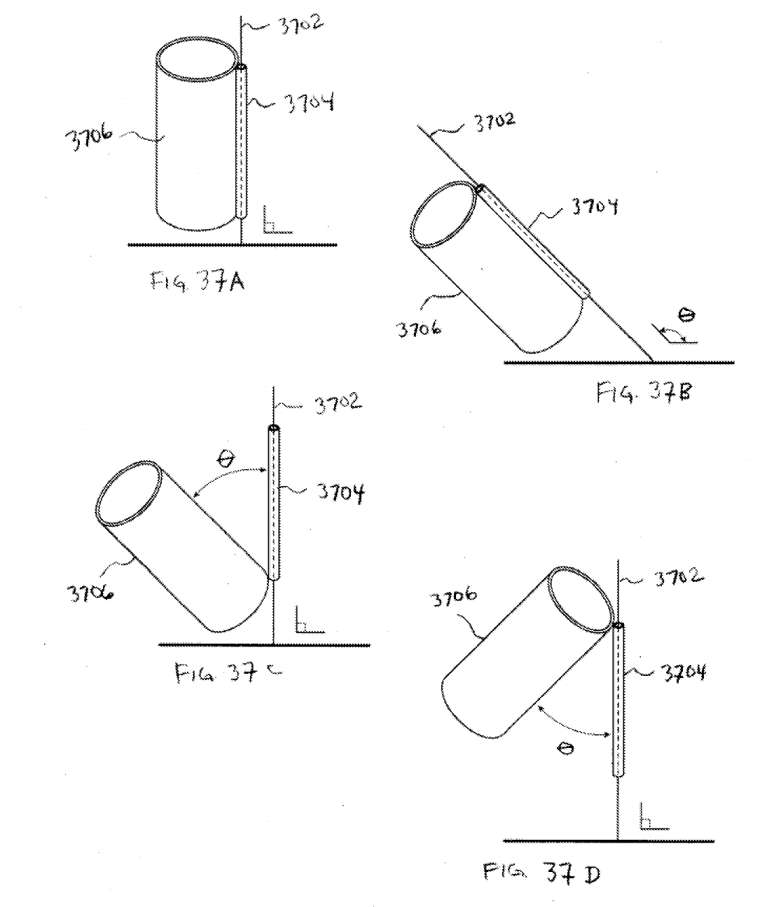

Referring now to FIGS. 28-31, light input connector 28312C surrounds light input cylinder 28312 which may be divided into multiple input arms such as arms 28311 and 28313 that then direct light into illumination waveguide 28310. Input arms 28311 and 28313 may assume any suitable shape and cross-sections depending on the optical design goals, such as the multi-radius arms with rectangular cross-section shown or straight sections (no radius) or angle rotators, etc. Also shown is a clamp flange holder 28314 that serves to support input connector 28312C and arms as well as providing a standard light connector 28312C over input cylinder 28312 (e.g., an ACMI or WOLF connector) and a flange 2314F at the top for attaching a clamp used to hold the entire structure in place once it is positioned relative to a surgical site in a body. A shelf or other similar light blocking structures may be added to the holder, extending over the input arms and or the upper tube edge as needed to help block any light that may escape these structures that might shine up into the user's eyes. Circumferential light extraction structures 28316 are shown at the bottom, distal end 28318, of the tube. In the section view of FIG. 29, vertical light disruption structures or facets 29276F are shown on the inside wall of the tube.

Illuminated cannula 28310 of FIG. 28 includes clamp adapter 28314 that also support light coupling 28312C for introducing light energy into cannula 28310. The relative orientation of the clamp adapter and the light coupling as shown enables the clamp adapter to operate as a shield to prevent any misdirected light shining into the eyes of anyone looking into bore 28310B of the cannula, but the clamp adapter and light coupling may adopt any suitable orientation.

FIG. 29 illustrates vertical facets 29276F within the distal end for disrupting the light spiraling within the waveguide. Circumferential light extraction structures 28316 may include stepped facets such as facets 28316F and risers such as riser 28316R on the outside tube wall 29310W. The “riser” section of the stepped facet section 28316R is angled so that it may slide against tissue without damaging the tissue. Steps may be uniform or non-uniform depending on the light directional control desired. The steps may be designed to direct light substantially inwards and toward the bottom of the tube or some distance from the bottom of the tube, or they may be designed to direct light toward the outside of the tube, or both.

Circumferential light extraction structures such as structures 28316 may be facets or may be other geometries, such as parabolas. Circumferential light extraction structures coupled with light directing structures that provide circumferentially distributed light to the extraction structures provide circumferential illumination. Since tools entering the interior of the tube now have light shining on them from all sides, the tools do not cast any shadows within the cone of illumination emitted by the cannula. The circumferential illumination from a cylindrical waveguide creates a generally uniform cone of light that minimizes shadows, e.g., from instruments, creating substantially shadowless illumination in the surgical field below the tubular waveguide.

COP Cannula 28310 of FIGS. 30-31 is illustrated without clamp flange/holder 28314 in place. Input arms 28311 and 28313 above are offset above proximal surface 30319 by a distance 30320 and end in angled reflector surface 30321 that partially extends down distance 30322 into the tube wall. The offset controls the light entering waveguide 28310 and restricts light entering to input structure 30323. Reflector surface 30321 serves to direct light orthogonally from the horizontal input and down into the tube wall, also causing the light to spread around the circumference of the tube wall by the time the light reaches the distal or lower part of the tube. Reflector surfaces such as surface 30321 may be a flat surface, an arced surface, or a series of interconnected surfaces and may also end at the top of the tube wall. Reflector surface 30321 may be treated, e.g., a reflective or metallized coating or an applied reflective film, to enhance reflection.

Air gaps may be used to isolate the light-conducting pathway in any suitable connector. Waveguide 28310 of FIG. 32 includes male connector 32324C that has been integrated with waveguide tube wall 29310W via bracket 32325. This allows connector 32324C to be molded with the waveguide and not attached as a separate part, such as standard light connector 28312C shown in FIG. 28. A separate connector introduces tolerance concerns into the system that may result in reduced coupling efficiency between a fiber optic cable output and waveguide input 32326 because the two parts may not be aligned correctly. Molding the connector and the waveguide input as one piece substantially reduces the chance of misalignment and thereby increases coupling efficiency.

FIG. 33 is a front view looking into input 32326 of connector 32324C. Air gaps 32327 are maintained around waveguide input 32326 to isolate the light-conducting pathway. One or more small zones of contact such as contact zone 32327C may be maintained, essentially bridging connector 32324C and input 32326 with a small amount of material, to add strength and stability to the system while resulting in minimum light loss in the contact zone.

COP Waveguide 34330 of FIGS. 45-46 may be split open during surgery to permit greater access to the surgical field. Waveguide 34330 is preferably formed of cyclo olefin polymer. Light input channels 35331 and 34333 may be split and fed through a “Y”. Waveguide 34330 is fully split front and back from the top to about ½-⅔ of tube by slots 34334 and 35336. Alternatively, a waveguide may be split all the way to lower portion 34330L. Lower portion 34330L is scored inside and out with scoring such as score 34337. The scoring operates to redirect light that may be trapped circling the tube. Bottom element 35340 may also be a COP element and is pre-split in half along edge 35341 and may be glued or otherwise secured in a waveguide such as COP waveguide 34330. The generally planar shape of element 35340 permits viewing through bottom element 35340 and allows light to shine through. Alternatively, element 35340 may also adopt any other suitable geometry such as rounded to form a lens. Because of the interface with the tube along edge 35342 very little light is conducted into element 35340. Hole 35343 enables a surgical screw or other suitable connector to engage through bottom element 35340 of waveguide 34330 to a surgical site. Splitting waveguide 34330 and bottom 35340 frees the waveguide elements from a connector through hole 35343, and permits the waveguide elements to be removed from the surgical site. While at least one light extraction structure is preferably located in lower portion 35330L on each tube half, the at least one extraction structure may be located on only one half or may be located further up the tube, e.g., near the end of split 34334 and or split 34336.

COP waveguide 36344 in FIG. 36 has reflector face 36345 extending down the side of waveguide 36344 opposite light input 36346, effectively removing material 36347. Extended reflector face 36345 serves to direct light circumferentially around the tube wall. This opens up the waveguide to provide improved access to the surgical space. In addition, it offers the opportunity to replace removed material 36347 with more durable material to improve strength and or provide a second clamp flange holder and or to provide mounting for other devices, such as a CCD camera.

Methods of Use

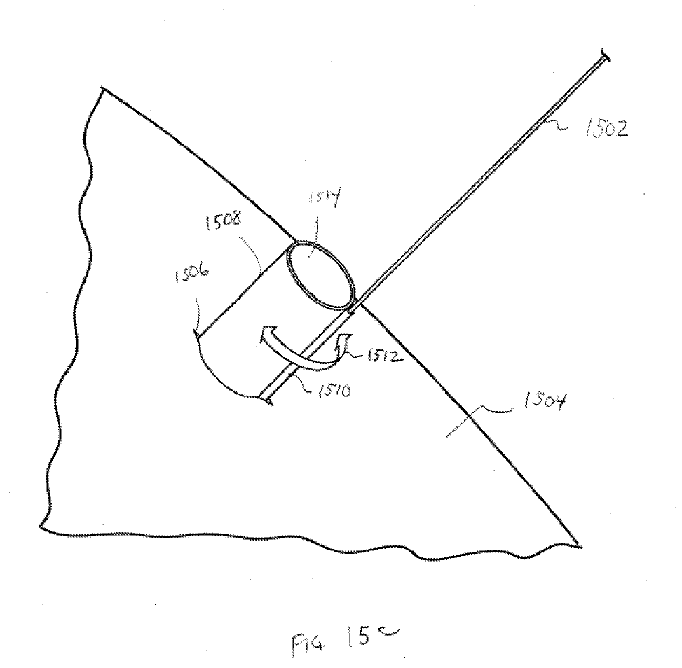

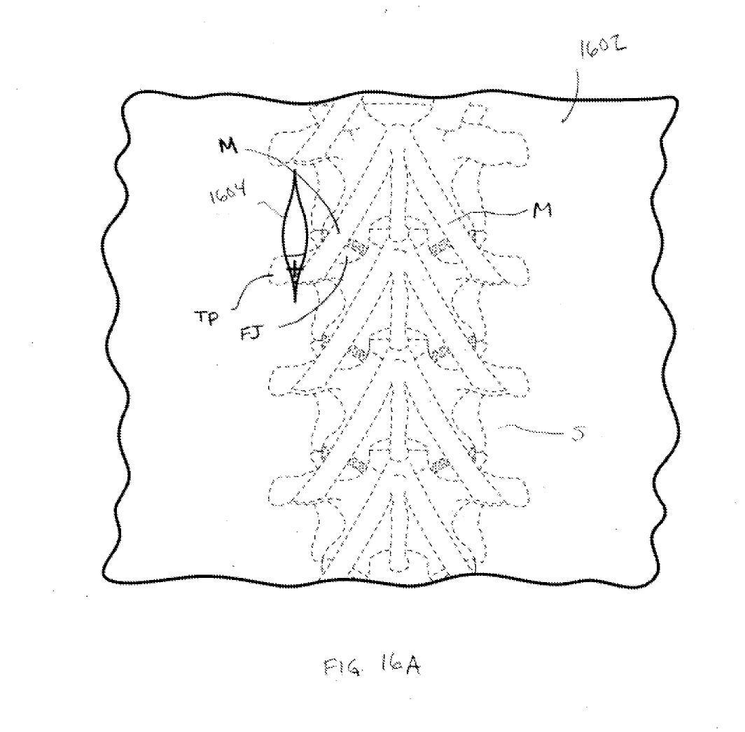

FIGS. 15A-15C illustrate an exemplary method of using the retractors described herein. In FIG. 15A, an incision is made into a patient's skin 1504 and an anchoring element 1502 is then advanced through the incision into the tissue and then anchored into position. The anchoring element may be a guidewire, a pedicle screw tower, or other anchor. The anchoring element may be secured to the tissue or bone. In FIG. 15B, any of the retractor embodiments described in this specification may then be coupled to the anchoring element and then advanced over the anchoring element into the incision. In FIG. 15B, channel 1510 is slidably advanced over the anchoring element 1502, here a guidewire. Once the retractor 1508 is advanced into the incision 1506, it will retract tissue away thereby creating an open surgical field that can be accessed by the bore 1514 of the retractor 1508. Additionally, the retractor may then be actuated by rotating it around the anchor element 1502 to move tissue and adjust the position of the surgical field. FIG. 15C illustrates rotation of the retractor around the guidewire 1502. Thus, the retractor will be rotated eccentrically about the pivot point created by the anchoring element. In other embodiments disclosed above, the retractor may be actuated into its expanded configuration in order to retract tissue. In addition to creating access to the surgical field, the bore may be used to deliver photocure agents such as cement or other therapeutic agents (e.g. bone morphogenetic proteins) to the treatment site.

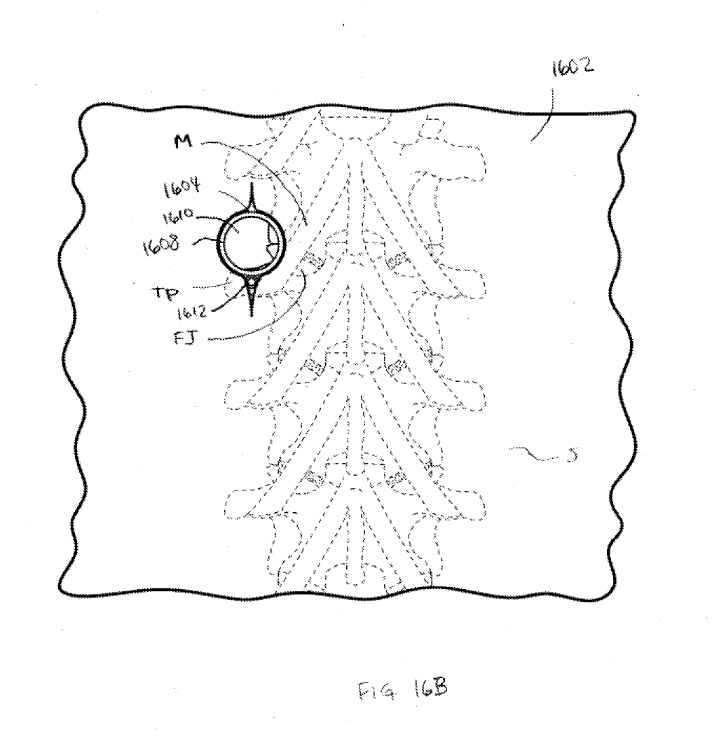

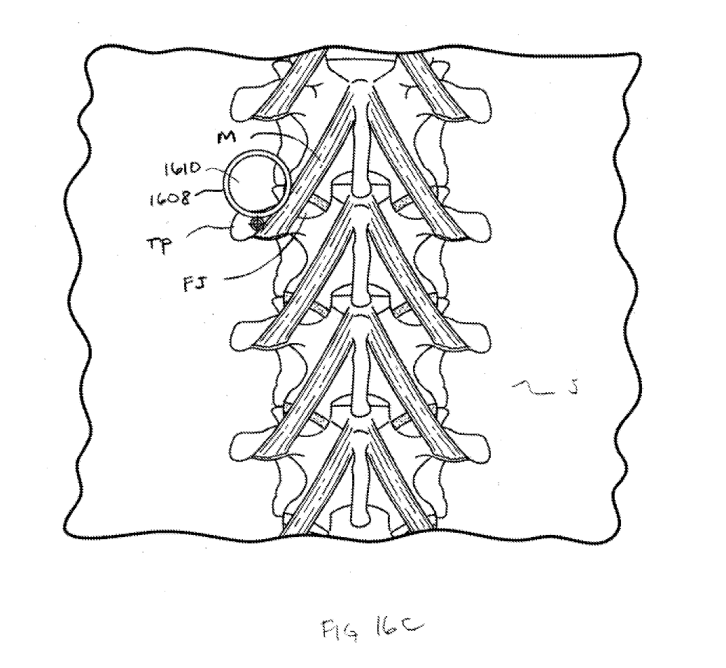

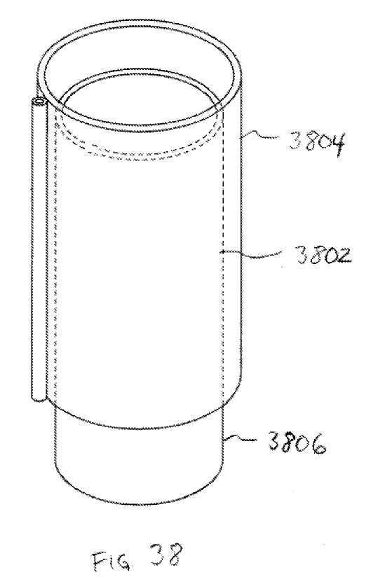

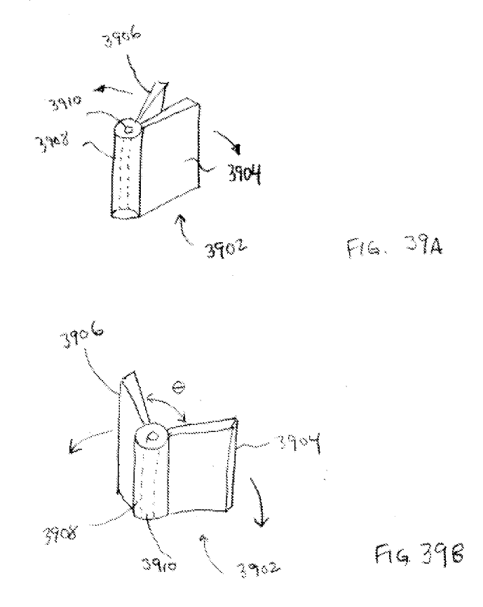

FIGS. 16A-16E illustrate another exemplary method of using the retractors described herein in spinal surgery. In FIG. 16A an incision 1604 is made through a patient's skin 1602 to access the spine S. Various muscles such as the paraspinal/multifidi muscle M often obstruct a surgeon's access to the facet joint FJ and adjacent areas such as the transverse processes TP. An anchoring element such as a guidewire or pedicle screw tower (not illustrated) is then anchored to the bone. In FIG. 16B, the channel 1612 on any of the retractors disclosed herein is then coupled to the anchoring element and then the retractor is advanced over the anchoring element into the incision 1604. This retracts tissue and creates a surgical field that can be accessed through the bore 1610 of the retractor 1608 and allows photocure and therapeutic agents to be delivered to the treatment site. FIG. 16C is the same view as FIG. 16B except with the skin removed for convenience of illustrating the anatomy. In FIG. 16D the retractor is rotated thereby moving the paraspinal/multifidi muscle M out of the way and thus allowing a surgeon to access the facet joint FJ. In FIG. 16E, the retractor is rotated in the opposite direction thereby retracting tissue and allowing access to an adjacent area such as the transverse processes TP. While the exemplary methods illustrated generally show the retractor being positioned vertically and substantially perpendicular to the surgical field (e.g. FIG. 37A with retractor 3706, anchoring element 3702 and channel 3704), one of skill in the art will appreciate that the anchoring element and/or the retractor may approach the surgical field with an angled approach. For example, in FIG. 37B, the anchoring element 3702 may be disposed at an angle relative to the surgical field and thus retractor 3706 and channel 3704 are slidably advanced over the anchoring element in parallel, but angled θ relative to the surgical field. FIG. 37C illustrates another exemplary embodiment where the anchoring element 3704 is positioned generally perpendicular to the surgical field and the channel 3702 is slidably disposed thereover in parallel, but the retractor is adjustable relative to the channel, thus the retractor can be adjustably angled θ relative to the anchoring element and the surgical field. FIG. 37D illustrates a similar embodiment to that of FIG. 37C, with the major difference being that instead a top or proximal end of the retractor pivoting relative to the anchoring element, the bottom or distal portion of the retractor pivots relative to the anchoring element.

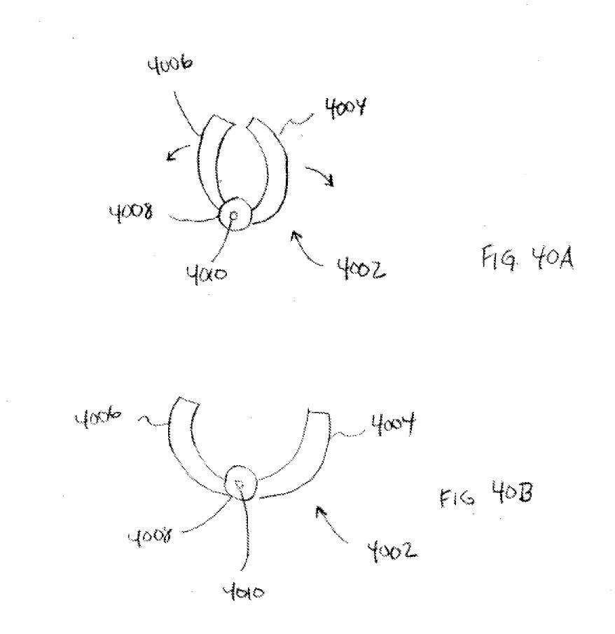

FIG. 38 illustrates still another embodiment of a tubular retractor 3802 which may be any of the embodiments disclosed herein with an outer sleeve 3804 disposed thereover. The outer sleeve 3804 may be metal, a polymer or another material. The outer sleeve prevents direct contact between the retractor and the blood and tissue in the surgical field. This is advantageous especially when the tubular retractor is also a waveguide because avoiding contact with blood or tissue and the waveguide helps to minimize light leakage from the waveguide. In preferred embodiments, a distal portion 3806 of the retractor/waveguide is exposed from the sleeve 3804 in order to allow light to be extracted and directed to the surgical field. In alternative embodiments, the sleeve may be placed on the inside of the tubular retractor, or a sleeve may be placed on the inside and outside of the tubular retractor. Other claddings or coatings may be applied to the retractor, the sleeve, or both in order to promote total internal reflection and minimize light loss. Films may also be applied to the retractor to facilitate extraction of light. The channel for receiving the anchoring element may be disposed on the outer surface of the sleeve or along any of the other surfaces of the sleeve or retractor.

Expandable Retractors

FIGS. 39A-39B illustrate an exemplary embodiment of an expandable retractor 3902. The retractor 3902 may be inserted through an incision in a collapsed, low profile configuration and then expanded to retract tissue and provide access to a surgical field. The retractor 3902 includes a first and second retractor blade 3904, 3906 that are pivotably coupled to a hinge 3908. The hinge 3908 may have a central bore 3910 extending therethrough so that the retractor 3902 may be coupled to an anchoring element such as the guidewire, pedicle screw, or pedicle screw tower previously described above, or coupled to other anchoring elements. In this exemplary embodiment the retractor blades are flat planar blades having a rectangular shape, but they may be any of the shapes disclosed herein or known in the art. During insertion, the retractor blades 3904, 3906 are in a collapsed configuration such that they engage one another in a flat planar low profile as seen in FIG. 39A. After insertion into the incision, the retractor blades may be pivoted about the hinge into an expanded configuration thereby retracting tissue. The retractor blades may be opened to any desired angle θ and then locked into the expanded position using locking mechanisms known in the art (e.g. ratchets, detents, set screws, etc.).

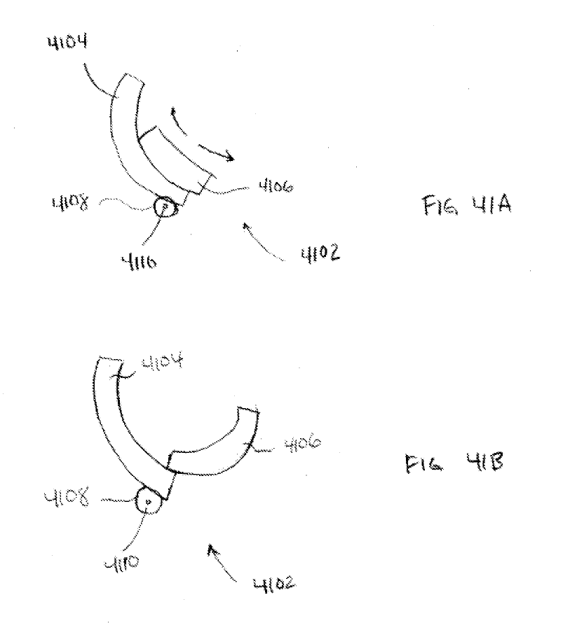

FIGS. 40A-40B illustrate another exemplary embodiment of an expandable retractor 4002. In this embodiment, the retractor includes two arcuate blades 4004, 4006 coupled together with a hinge 4008 having a central bore 4010 for coupling with any of the anchoring elements disclosed herein. The retractor blades are pivoted inward toward one another during insertion into an incision as seen in FIG. 40A. Once in the incision, the retractor blades 4004, 4006 are then pivoted outward away from one another to form a semi-circle or other curved shape, thereby retracting tissue as seen in FIG. 40B. The blades may be opened any amount depending on the desired amount of retraction and then they may be locked into position as described above.

FIGS. 41A-41B illustrate yet another example of an expandable retractor. The retractor 4102 includes two retractor blades 4104, 4106 that are slidably engaged with one another. A ratchet mechanism, detents, linear slide mechanism, or other mechanisms known in the art may be used to operably couple the two blades together and allow them to slide relative to one another. During insertion, the retractor 4102 is inserted into the incision in its low profile configuration with both of blades slidably advanced inward toward one another as seen in FIG. 41A. Once positioned in the incision, the blades may be slidably expanded relative to one another into the expanded configuration seen in FIG. 41B. Expanding the blades lengthens the region of tissue contact and hence the amount of tissue retraction. The embodiment in FIGS. 41A-41B show the blades expanding into an arcuate configuration, although the blades may be expanded into other configurations, such as a straight line, semi-circle, etc. A coupling element 4108 with a central bore 4110 for anchoring to an anchoring element may also be coupled to any portion of any of the retractor blades.

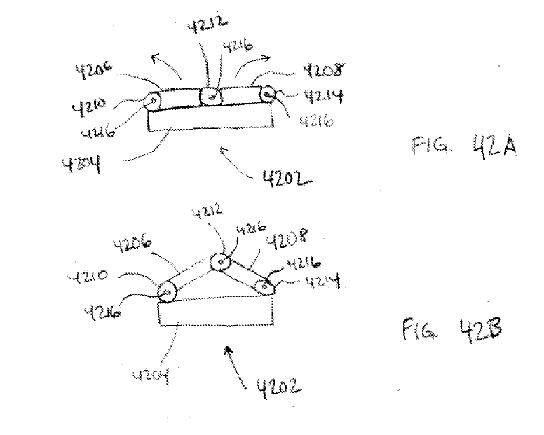

FIGS. 42A-42B illustrate yet another exemplary embodiment of an expandable retractor. The retractor 4202 has a low profile collapsed configuration seen in FIG. 42A and an expanded configuration seen in FIG. 42B. The retractor 4202 includes three retractor blades 4204, 4206, 4208 that are coupled together with hinges 4210, 4212, 4214. The hinges 4210, 4212, 4214 may have central bores 4216 extending therethrough so that the hinges may be coupled to an anchoring element such as a guidewire, pedicle screw or pedicle screw tower. In use, the retractor 4202 is inserted into the incision in the collapsed configuration with the retractor blades folded inward against one another. After insertion into the incision, the retractor blades may be actuated into an expanded configuration by pivoting them relative to their hinge. The expanded configuration may form a triangular shape or other polygon shape, with tissue retracted away from the center of the triangle or polygon. Once expanded, the retractor may be locked into the open position with a locking mechanism such as a set screw, detents, ratchets, etc.

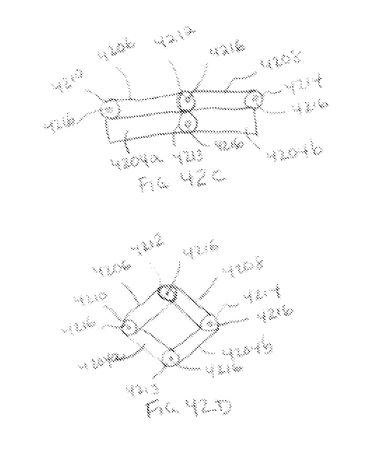

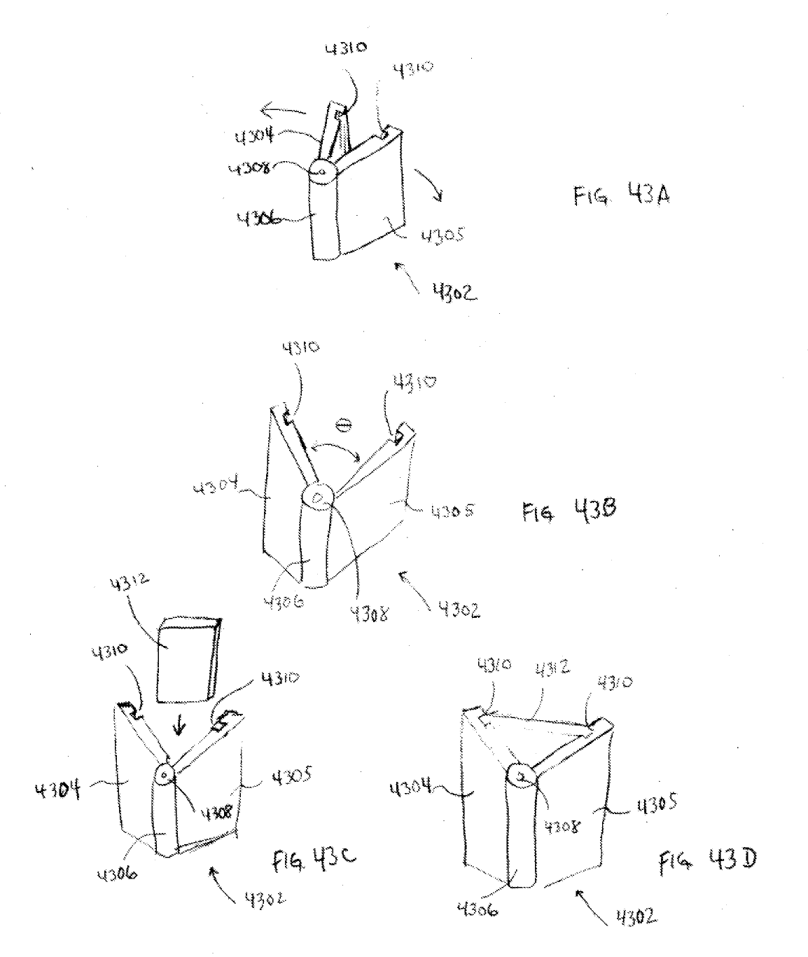

FIGS. 42C-42D illustrate an alternative embodiment similar to that in FIGS. 42A-42B. The major difference being that in this embodiment, the device has four retractor blades and an additional hinge. Retractor blades 4204a, 4204b are coupled together by hinge 4213. Thus, when expanded, the blades open up to to form a diamond shape as seen in FIG. 42D. Any number of blades may be hingedly coupled together to form a polygon in the expanded configuration. The larger the number of blades, the more closely the expanded configuration will be able to form a smoother, circular shape, or a symmetric or non-symmetric polygon. FIGS. 43A-43D illustrate another exemplary embodiment of an expandable retractor 4302. The retractor 4302 is similar to the hinged embodiment in FIGS. 39A-39B with the major difference being that it receives another retractor blade 4312 in slots 4310 to form a triangular region or other polygon shaped region of retraction and lock the retractor into the expanded configuration. The retractor includes two blades 4304, 4305 coupled together with a hinge 4306 having a central bore 4308 for engaging an anchoring element. Each blade includes a slotted region or elongate channel 4310. The retractor 4302 is inserted in a low profile configuration with both blades folded inward toward one another as illustrated in FIG. 43A. Once inserted into the incision, the blades may be pivoted outwardly to any desired angle θ as seen in FIG. 43B. A third retractor blade 4312 is then slidably engaged with the channels 4310 to lock the retractor into position as seen in FIGS. 43C and 43D.

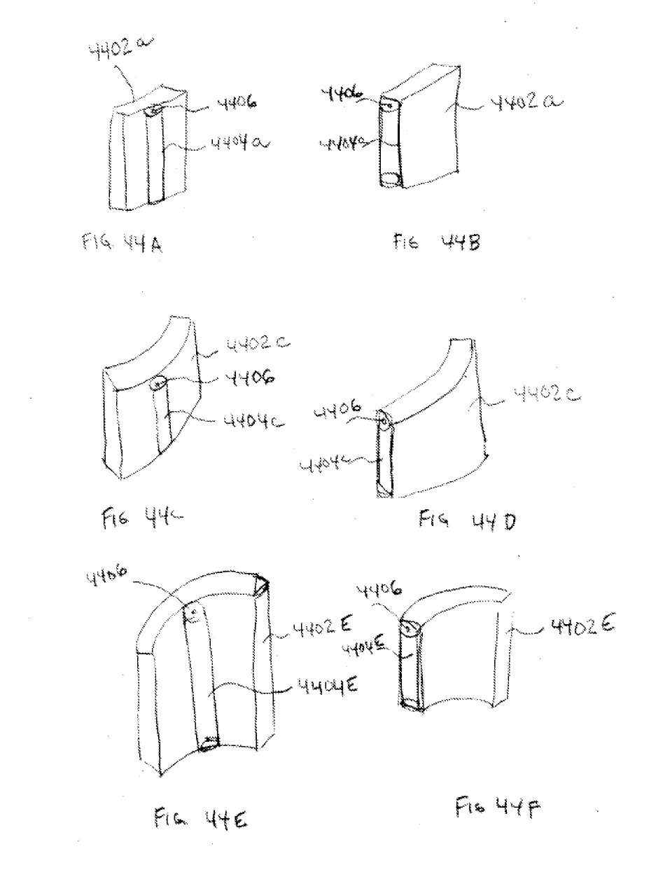

The expandable retractors described above, as well as any of the retractors disclosed herein may have blades in any number of configurations. Additionally, the coupling element may be positioned in any number of locations along the blade, depending on the desired actuation pattern and anatomy being treated. For example, FIG. 44A illustrates a flat rectangular and planar retractor blade 4402a with the coupling element 4404a on either a front or rear surface of blade. The coupling element may be centered or off-center and may include a central bore for receiving a guidewire or other anchoring element. FIG. 44B illustrates a variation of the embodiment in FIG. 44A wherein the coupling element 4404a has been moved to an end or edge of the retractor blade.

FIG. 44C illustrates another embodiment where the retractor blade 4402c is arcuate and has a concave front surface and a convex outer surface with the coupling element on the outer surface. FIG. 44D illustrates the same embodiment except with the coupling element moved to an end or edge of the retractor blade. FIG. 44E illustrates another similar retractor blade 4402e, except this time with the coupling element 4404e on the front concave surface of the blade. FIG. 44F shows the coupling element 4404e moved to the end or edge of the retractor blade.

Any of the other features described herein (e.g. illumination) may be combined with or substituted with any of the retractor embodiments disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A positionable surgical retractor, said retractor comprising:
a retractor blade having a first surface, a second surface and a wall extending therebetween, wherein the first surface is adapted to engage and retract tissue away from a surgical field, and wherein the second surface is opposite the first surface; and
a coupling element coupled to the retractor blade or disposed in the wall, wherein the coupling element is adapted to be coupled to an anchor element, and wherein the retractor is positionable relative to the anchor so as to engage and retract the tissue.

2. The retractor of claim 1, wherein the retractor blade comprises an elongate tubular body.

3. The retractor of claim 2, wherein the elongate tubular body comprises a cylinder.

4. The retractor of claim 1, further comprising an illumination element coupled thereto, the illumination element adapted to illumination the surgical field.

5. The retractor of claim 1, wherein the retractor blade is a waveguide, the waveguide comprising a light input portion, light extraction features adjacent a distal end of the retractor blade, and a light transmitting portion disposed therebetween, wherein light is input into the retractor blade from the light input, and wherein light is transmitted through the light transmitting portion by total internal reflection, and wherein light is extracted and directed from the retractor blade to the surgical field by the light extraction features.

6. The retractor of claim 5, wherein the light extraction features comprise a plurality of facets, prisms, or lenses.

7. The retractor of claim 1, further comprising a flanged region adjacent a distal end of the retractor, the flanged region adapted to prevent tissue from sliding off the retractor.

8. The retractor of claim 1, further comprising a contoured distal end adapted to conform to anatomy in the surgical field and wherein the contoured distal end allows movement of the retractor over the anatomy.

9. The retractor of claim 1, wherein the coupling element comprises a snap fitting adapted to engage the anchor.

10. The retractor of claim 1, wherein the coupling element comprises a tubular channel.

11. The retractor of claim 10, wherein the tubular channel is disposed on the first surface or the second surface of the retractor blade.

12. The retractor of claim 10, wherein the tubular channel is disposed in the wall of the retractor blade.

13. The retractor of claim 1 further comprising a handle coupled with the retractor blade, the handle adapted to facilitate actuation of the retractor blade.

14. The retractor of claim 1, wherein the retractor blade has an adjustable length.

15. The retractor of claim 1, wherein the retractor blade has an adjustable width or sweep.

16. The retractor of claim 1, wherein the retractor blade comprises a plurality of retractor blades hingedly coupled together, wherein the plurality of retractor blades have a collapsed configuration for insertion into an incision and an expanded configuration for retracting tissue in the surgical field, and wherein the plurality of retractor blades are adjacent one another in the collapsed configuration, and wherein the plurality of retractor blades are actuated away from one another in the expanded configuration.

17. The retractor of claim 16, wherein the plurality of retractor blades form a semi-circle in the expanded configuration.

18. The retractor of claim 16, wherein the plurality of retractor blades comprises three retractor blades hingedly coupled together, and wherein the plurality of retractor blades form a polygon in the expanded configuration.

19. The retractor of claim 18, wherein the polygon comprises a triangle or a diamond.

20. The retractor of claim 16, wherein the plurality of retractor blades comprise two retractor blades each having a slot for slidably receiving a third retractor blade, the third retractor blade holding the two slotted retractor blades in the expanded configuration.

21. A system for retracting tissue in a surgical field, said system comprising:
the surgical retractor of claim 1; and
the anchoring element.

22. The system of claim 21, wherein the anchoring element comprises a guidewire.

23. The system of claim 21, wherein the anchoring element comprises a pedicle screw tower.

24. The system of claim 21, wherein the anchoring element comprises spinal instrumentation.

25. A method of retracting tissue in a surgical field, said method comprising:
anchoring an anchoring element in the surgical field;
coupling a retractor blade to the anchoring element;
disposing the retractor blade in the surgical field;
actuating the retractor blade about the anchoring element; and
retracting the tissue in the surgical field.

26. The method of claim 25, wherein the anchoring element comprises a guidewire and anchoring the anchoring element comprises anchoring the guidewire in the surgical field.

27. The method of claim 25, wherein the anchoring element comprises spinal instrumentation, and anchoring the anchoring element comprises anchoring the spinal instrumentation in the surgical field.

28. The method of claim 25, wherein the anchoring element comprises a pedicle screw tower, and anchoring the anchoring element comprises anchoring the pedicle screw tower in the surgical field.

29. The method of claim 25, wherein coupling the retractor blade comprises slidably engaging the retractor blade with the anchoring element.

30. The method of claim 25, wherein coupling the retractor blade comprises snap fitting the retractor blade with the anchoring element.

31. The method of claim 25, wherein coupling the retractor blade comprises releasably engaging the retractor blade with the anchoring element.

32. The method of claim 21, wherein disposing the retractor blade in the surgical field comprises sliding the retractor blade over the anchoring element into the surgical field.

33. The method of claim 21, wherein actuating the retractor blade comprises rotating the retractor blade about the anchoring element.

34. The method of claim 33, wherein rotating the retractor blade comprises rotating the retractor blade eccentrically about the anchoring element.

35. The method of claim 22, wherein retracting tissue in the surgical field comprises retracting a muscle.

36. The method of claim 35, wherein the muscle comprises a multifidi or paraspinal muscle.

37. The method of claim 22, wherein retracting tissue comprises exposing a facet joint.

38. The method of claim 22, wherein the retractor blade comprises a waveguide, the method further comprising illuminating the surgical field with light from the waveguide.

39. The method of claim 22, wherein the retractor blade comprises an illumination element, the method further comprising illuminating the surgical field with light from the illumination element.

40. The method of claim 25, wherein the retractor blade comprises a plurality of retractor blades disposed adjacent one another in a collapsed configuration, and the method further comprises actuating the plurality of retractor blades from the collapsed configuration into an expanded configuration wherein the plurality of retractor blades are actuated away from one another.

41. The method of claim 40, further comprising locking the plurality of retractor blades in the expanded configuration.
